(19) United States
(12) Patent Application Publication
(10) Pub. No.: US 2002/0028488 A1
(43) Pub. Date: Mar. 7, 2002

(54) Transgenic avian species for making human and chimeric antibodies

(76) Inventors: Sujay Singh, San Diego, CA (US); Peter Dias, Carlsbad, CA (US)

Correspondence Address: David R. Preston, Suite 205, 12625 High Bluff Drive, San Diego, CA, 92130, US

(21) Appl. No.: 09/884,579

(22) Filed: Jun. 18, 2001

Related U.S. Application Data
(60) Provisional application No. 60/212,456, filed on Jun. 19, 2000.

(51) Int. Cl. A01K 67/027; C12P 21/04; C07K 16/00

(52) U.S. Cl. 435/70.21; 800/19; 530/388.1

(57) ABSTRACT

The present invention provides methods for producing exogenous and chimeric antibodies in avians. One aspect of the present invention is a method of producing avians or avian cells lacking endogenous immunoglobulin light chain and heavy chain loci, or portions thereof, and having at least a portion of at least one exogenous immunoglobulin locus. The present invention provides a method for obtaining an avian cell with a deletion in an endogenous immunoglobulin locus by by introducing a targeting construct comprising two regions of sequences which are homologous to the 5′ and 3′ flanking sequences of the region to be deleted in the wild-type locus. In addition, the invention provides methods for inserting exogenous immunoglobulin gene loci into the genome of an avian cell. A second aspect of the invention is the generation of transgenic avian species or transgenic avian cells for producing chimeric antibodies. The avian host is characterized by: (1) being incapable of producing endogenous immunoglobulin; and (2) having at least a portion of an exogenous immunoglobulin locus comprising at least one immunoglobulin constant region or portion thereof. Specific binding proteins with xenogenic regions can be produced in a viable avian host by immunization of the avian host with an appropriate immunogen. Another aspect of the invention is the isolation of antibody-producing cells from a transgenic avian of the present invention that has been immunized with an antigen of interest. The cells can be immortalized for the production of antibody in culture. The immortalized cells can be used for the isolation of cDNAs encoding immunoglobulin heavy and light chains or portions thereof. The cDNAs can be reintroduced to cell lines, including mammalian cell lines for efficient production of monoclonal antibodies. The cDNAs can optionally be mutated or altered, for example, such that they encode higher avidity antibodies or chimeric immunoglobulin molecules, prior to reintroduction into cell lines.

This application claims benefit of priority to U.S. provisional application 60/212,456, filed Jun. 19, 2000, herein incorporated by reference.

### TECHNICAL FIELD

The present invention generally relates to transgenic avian species such as chickens, that are useful for making chimeric antibodies, human antibodies, or modified antibodies.

### BACKGROUND

Monoclonal antibodies are useful in analyte detection, purifications, diagnosis and therapy. Because of their ability to bind to a specific epitope, they can be uniquely used to identify molecules carrying that epitope or may be directed, by themselves or in conjunction with another moiety, such as a cytotoxic or radioactive moiety, to a specific site for diagnosis or therapy.

The basic immunoglobulin (Ig) structural unit in vertebrate systems is composed of two identical “light” polypeptide chains (approximately 23 kDa), and two identical “heavy” chains (approximately 53 to 70 kDa). The four chains are joined by disulfide bonds in a “Y” configuration, and the “tail” portions of the two heavy chains are bound by covalent disulfide linkages when the immunoglobulins are generated either by hybridomas or by B cells.

A schematic of the general antibody structure is shown in FIG. 1. The light and heavy chains are each composed of a variable region at the N-terminal end, and a constant region at the C-terminal end. In the light chain, the variable region (termed “V_{L} J_{L}”) is the product of the recombination of a V_{L} gene to a J_{L} gene. In the heavy chain, the variable region (V_{H} D_{H} J_{H}) is the product of recombination of first a D_{H} and a J_{H} gene, followed by a D_{H} J_{H} to V_{H} recombination. The V_{L} J_{L} and V_{H} D_{H} J_{H} regions of the light and heavy chains, respectively, are associated at the tips of the Y to form the antibody's antigen binding domain and together determine antigen binding specificity.

The (C_{H}) region defines the antibody's isotype, i.e., its class or subclass. Antibodies of different isotypes differ significantly in their effector functions, such as the ability to activate complement, bind to specific receptors (Fc receptors) present on a wide variety of cell types, cross mucosal and placental barriers, and form polymers of the basic four-chain IgG molecule.

Antibodies are categorized into “classes” according to the C_{H} type utilized in the immunoglobulin molecule (IgM, IgG, IgD, IgE, IgA, or IgY). There are at least five types of C_{H} genes (C mu, C gamma, C delta, C epsilon, and C alpha), and some species (including humans) have multiple C_{H} subtypes (e.g., C gamma₁, C gamma₂, C gamma₃, and C gamma₄ in humans). There are a total of nine C_{H} genes in the haploid genome of humans, eight in mouse and rat, and several fewer in many other species. In contrast, there are normally only two types of light chain constant regions (C_{L}), kappa and lambda, and only one of these constant regions is present in a single light chain protein (i.e., there is only one possible light chain constant region for every V_{L} J_{L} produced). Each heavy chain class can be associated with either of the light chain classes (e.g., a C_{H} gamma region can be present in the same antibody as either a kappa or lambda light chain).

A process for the immortalization of B cell clones producing antibodies of a single specificity has been developed involving fusing B cells from the spleen of an immunized mouse with immortal myeloma cells. Single clones of fused cells secreting the desired antibody can then be isolated by drug selection followed by immunoassay. These cells were given the name “hybridoma” and their antibody products termed “monoclonal antibodies.”

The use of monoclonal antibodies as therapeutic agents for human disease, for diagnostics, and for purification of antigens requires the ability to produce large quantities of the desired antibody. One approach to increased production was simply to scale up the culture of hybridoma cells. Although this approach is useful, it is limited to production of that antibody originally isolated from the mouse. In the case where a hybridoma cell produces a high affinity monoclonal antibody with the desired biological activity, but has a low production rate, the gene encoding the antibody can be isolated and transferred to a different cell line with a high production rate.

Recombinant DNA techniques have been used for production of heterologous proteins in transformed host cells, particularly mammalian cells. Generally, the produced proteins are composed of a single amino acid chain or two chains cleaved from a single polypeptide chain. More recently, multichain proteins such as antibodies have been produced by transforming a single host cell with DNA sequences encoding each of the polypeptide chains and expressing the polypeptide chains in the transformed host cell.

In some cases it is desirable to retain the specificity of the original monoclonal antibody while altering some of its other properties. For example, a problem with using murine antibodies directly for human therapy is that antibodies produced in murine systems may be recognized as “foreign” proteins by the human immune system, eliciting a response against the antibodies. A human anti-murine antibody (HAMA) response results in antibody neutralization and clearance and/or potentially serious side-effects associated with the anti-antibody immune response. Such murine-derived antibodies thus have limited therapeutic value.

One approach to reducing the immunogenicity of murine antibodies is to replace the constant domains of the heavy and light chains with the corresponding human constant domains, thus generating human-murine chimeric antibodies. Human-murine chimeric antibodies are generally produced by cloning the DNA sequences encoding the antibody variable regions and/or constant regions, combining the cloned sequences into a single construct encoding all or a portion of a functional chimeric antibody having the desired variable and constant regions, introducing the construct into a cell capable of expressing antibodies, and selecting cells that stably express the chimeric antibody.

In another approach, complementarity determining region (CDR)-grafted humanized antibodies have been constructed by transplanting the antigen binding site, rather than the entire variable domain, from a rodent antibody into a human antibody. Transplantation of the hypervariable regions of an antigen-specific mouse antibody into a human heavy chain gene has been shown to result in an antibody retaining antigen-specificity with greatly reduced immunogenicity in humans.

While the resulting chimeric partly xenogeneic antibody is in some aspects more useful than using a fully xenogeneic antibody, it still has a number of disadvantages. The identification, isolation and joining of the variable and constant regions requires substantial work. In addition, the joining of a constant region from one species to a variable region from another species may change the specificity and affinity of the variable regions, so as to lose the desired properties of the variable region. Also, there are framework and hypervariable sequences specific for a species in the variable region. These framework and hypervariable sequences may result in undesirable antigenic responses.

It would therefore be more desirable to produce allogeneic antibodies for administration to a host by immunizing the host with an immunogen of interest. For primates, particularly humans, this approach is not practical. The human antibodies which have been produced have been based on the adventitious presence of an available spleen, from a host which had been previously immunized to the epitope of interest. While human peripheral blood lymphocytes may be employed for the production of monoclonal antibodies, these have not been particularly successful in fusions and have usually led only to IgM. Moreover, it is particularly difficult to generate a human antibody response against a human protein, a desired target in many therapeutic and diagnostic applications.

It is now possible to produce transgenic mice that are capable, upon immunization, of producing a full repertoire of human antibodies in the absence of endogenous immunoglobulin production. A method of making transgenic mice lacking endogenous heavy and light immunoglobulin chains, and having exogenous human immunoglobulin loci, such that the mice can produce fully humanized antibodies, is described in U.S. Pat. No. 5,939,598 issued Aug. 17, 1999; U.S. Pat. No. 6,114,598 issued Sep. 5, 2000; and U.S. Pat. No. 6,162,963 issued Dec. 19, 2000 to Kucherlapati et al. In addition, it has been described that the homozygous deletion of the antibody heavy chain joining region (J_{H}) gene in chimeric and germ-line mutant mice will result in the production of human antibodies upon antigen challenge. See, for example, Jakobovits et al. (1993) Proc. Natl. Acad. Sci. USA 90: 2551-2555 and Jakobovits et al. (1993) Nature 362: 255-258).

In some instance, however, the high degree of relatedness between mammalian proteins can make the generation of an antibody to a human protein, in for example, a mouse, difficult or impossible.

In the alternative antibodies or antibody fragments can be isolated from antibody phage libraries generated using the techniques described in McCafferty et al. (1990) Nature 348: 552-554, using the antigen of interest to select for a suitable antibody fragment. Clackson et al. (1991) 352: 624-628 and Marks et al. (1991) J. Mol. Biol. 22: 581-597 describe the isolation of murine and human antibodies, respectively, using phage libraries. Subsequent publications describe the production of high affinity (nanomolar range) human antibodies by chain shuffling (Mark et al. (1992) Bio Technol. 10: 779-783), as well as combinatorial infection and in vivo recombination as a strategy for constructing very large phage libraries (Waterhouse et al. (1993) Nuc. Acids Res. 21: 2265-2266).

For a given disease indication, one antibody isotype is likely to be greatly preferred over another. The preferred isotype may vary from one indication to the next. For example, to treat cancer it may be desirable that the binding of an antibody to a tumor cell result in killing of a tumor cell. In this case, an IgG1 antibody, which mediates both antibody-dependent cellular cytotoxicity and complement fixation, would be the antibody of choice. Alternatively, for treating an autoimmune disease, it may be important that the antibody only block binding of a ligand to a receptor and not cause cell killing. In this case, an IgG4 or IgG2 antibody would be preferred. Thus, even in a situation where a high affinity, antigen-specific, fully human antibody has been isolated, it may be desirable to re-engineer that antibody and express the new product in a different cell.

The cell type to be used for the production of antibodies will also affect the glycosylation pattern of the antibodies. Glycosylation differences in antibodies are generally confined to the constant domain and may influence the antibodies' structure (Weitzhandler et al. (1994) J. Pharm. Sci. 83: 1760; Wyss and Wagner (1996) Curr. Opin. Biotech. 7: 409-416; Hart (1992) Curr. Opin. Cell Biol. 4: 1017-1023) and function (Boyd et al. (1996) Mol. Immunol. 32: 1311-1318; Wittwer and Howard (1990) Biochem. 29;4175-4180). Although cells from mammals, particularly mice, have been used for the production of antibodies, chicken immunoglobulins have been found to contain sialylated oligosaccharides having N-acetylneuraminic acid and lack oligosaccharides with N-glycolylneuraminic acid, a pattern also seen for human immunoglobulins, whereas mouse, sheep, cows, goats, horses, and rhesus monkeys have different profiles of sialylated oligosaccharides (Raju et al. Glycobiology 10: 477-486 (2000)). Although a variety of studies have focused on mammalian cells as the source of allogenic antibodies, either in culture or in an organism, the use of avian cells has not received significant attention.

The yolk antibody class IgY has received some interest due to the relatively large concentration of IgY in the yolk of an avian egg. Although the IgY class is not allogenic to humans and thus is of limited value in a variety of applications (including in vivo diagnostics and therapeutics), it has recently been shown that human IgG and IgA produced in cells implanted in chickens can be deposited in the egg yolk (Mohammed et al. Immunotechnology 4: 115-25 (1998)). The avian species possess a variety of valued characteristics, including growth to high density under farming conditions and a reduced target for animal rights activists, probably due the lack of fur and relative unattractiveness of certain members of the avian species, such as chickens. The present invention addresses these needs and provides other benefits as well.

### BRIEF DESCRIPTION OF THE FIGURES

FIG. 1 is a schematic showing the basic immunoglobulin structure.

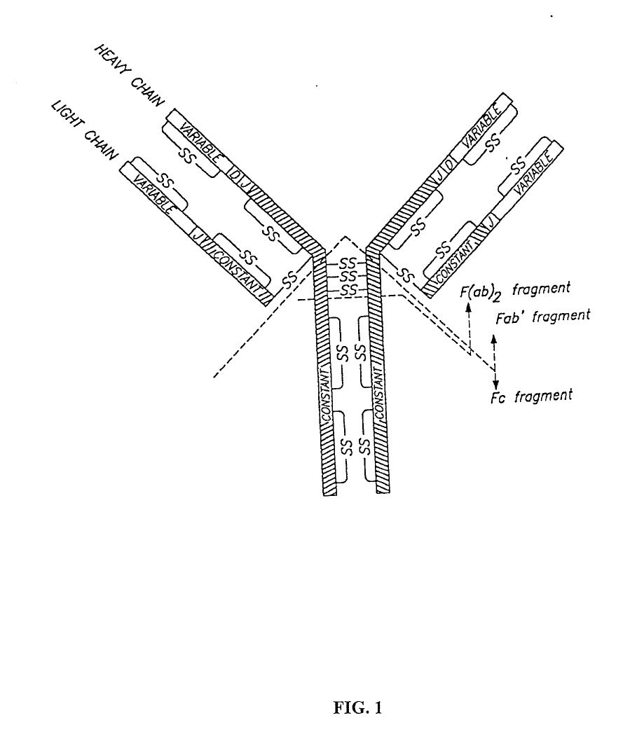

FIG. 2A is a schematic representation of a human immunoglobulin heavy chain locus and restriction fragments thereof.

FIG. 2B depicts a human heavy chain replacement YAC vector.

FIG. 3 is a schematic representation of the chicken heavy and light chain immunoglobulin loci.

FIG. 4 is a diagram of breeding strategy to obtain transgenic chickens lacking both endogenous immunoglobulin light chains and heavy chains.

### SUMMARY

The present invention recognizes that transgenic avian species, including chickens, ducks, geese, turkeys, and quails, can be engineered such that they can produce fully human antibodies, avian-human chimeric antibodies, or humanized antibodies. The present invention recognizes that immunization of avian species can be a useful way of producing antibodies that can recognize conserved epitopes on mammalian molecules which, because of self-tolerance, are not obtained by immunizing mammals such as mice. In one aspect, the present invention contemplates using avian species to produce large quantities of antibody that can readily be isolated from avians, including avian eggs.

One aspect of the present invention is a method of producing avians or avian cells lacking endogenous immunoglobulin light chain and heavy chain loci, or portions thereof, and having at least a portion of at least one exogenous immunoglobulin locus. The present invention provides a method for obtaining an avian cell with a deletion in a target locus which comprises modifying the genome of a cell containing the wild-type locus by introducing a targeting construct comprising two regions of sequences which are homologous to the 5′ and 3′ flanking sequences of the region to be deleted in said wild-type locus. The method further provides methods for gene disruption to disrupt expression of the avian heavy chain and light chain immunoglobulin loci. In addition, the invention provides methods for inserting exogenous immunoglobulin gene loci into the genome of an avian cell. The deletion or disruption of an endogenous immunoglobulin loci or portions thereof may or may not be achieved in the same step as insertion of an exogenous immunoglobulin loci or portions thereof. The method may further comprise culturing the modified cells in a medium containing a selectable agent and recovering cells containing said deletion or disruption and/or said insertion. The avian cells of the invention can be either primary cells or transformed cell lines, and may include any cell type, but are preferably B-lymphocytes, sperm cells, primordial germ cells, embryonic stem (ES) cells, or zygote cells.

A second aspect of the invention is the generation of transgenic avian species or transgenic avian cells for producing chimeric antibodies. The avian host is characterized by: (1) being incapable of producing endogenous immunoglobulin; and (2) having at least a portion of an exogenous immunoglobulin locus comprising at least one immunoglobulin constant region or portion thereof. In a preferred embodiment, the avian host will comprise at least one xenogeneic constant region or portion thereof capable of being spliced to a functional J region of an endogenous or exogenous immunoglobulin locus. This aspect can be achieved, at least in part, by employing homologous recombination at the immunoglobulin loci for the heavy and light chains. Specific binding proteins with xeonogenic regions can be produced in a viable avian host by immunization of the avian host with an appropriate immunogen.

Another aspect of the invention is the isolation of antibody producing cells from a transgenic avian of the present invention that has been immunized with an antigen of interest. The cells can be immortalized for the production of antibody in culture. Alternatively, the immortalized cells can be used for the isolation of cDNAs encoding immunoglobulin heavy and light chains or portions thereof. The cDNAs can be reintroduced to cell lines, including mammalian cell lines for efficient production of monoclonal antibodies. The cDNAs can optionally be mutated or altered, for example, such that they encode higher avidity antibodies or chimeric immunoglobulin molecules, prior to reintroduction into cell lines.

Other aspects, features, and advantages of the invention will become apparent from the following detailed description, and the claims.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, immunology, chemistry, microbiology, molecular biology, cell science and cell culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989), Current Protocols in Molecular Biology, edited by Ausubel et al., John Wiley and Sons (1998); Harlowe and Lane, Antibodies, a Practical Approach, Cold Spring Harbor, N.Y. (1989); Goding, J. W., Monoclonal Antibodies: Principals and Practice: Production and Application of Monoclonal Antibodies in Cell Biology, Biochemistry, and Immunology, 3^{rd} ed., Harcourt (Academic Press (1996); Ritter and Ladyman, Monoclonal Antibodies: Production, Engineering, and Clinical Applications, Cambridge University Press (1995)). Other methods relevant to the present invention may be found in U.S. Pat. Nos. 5,916,771, 5,939,598, and 5,998,209, herein incorporated by reference. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

“Isolated polynucleotide” refers to a polynucleotide of genomic, cDNA, PCR or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, DNA, RNA, recombinant RNA, or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source, for example, free of cellular proteins, or (4) is expressed by a cell from a different species, or (5) does not occur in nature.

“Polypeptide” is used herein as a generic term to refer to native protein, fragments, or analogs of a polypeptide sequence.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including viruses, that can be isolated from a source in nature and that has not been intentionally modified by man in the laboratory is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal binding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

“Polynucleotide” refers to a polymeric form of nucleotides of a least ten bases in length, either ribonucleotides or deoxyribonucleotides or a modified form of either type of nucleotide. The term includes single and double stranded forms of DNA or RNA.

“Genomic polynucleotide” refers to a portion of the genome.

“Active genomic polynucleotide” or “active portion of a genome” refer to regions of a genome that can be up-regulated, down-regulated or both, either directly or indirectly, by a biological process.

“Directly” in the context of a biological process or processes, refers to direct causation of a process that does not require intermediate steps, usually caused by one molecule contacting or binding to another molecule (the same type or different type of molecule). For example, molecule A contacts molecule B, which causes molecule B to exert effect X that is part of a biological process.

“Indirectly” in the context of a biological process or processes, refers to indirect causation that requires intermediate steps, usually caused by two or more direct steps. For example, molecule A contacts molecule B to exert effect X which in turn causes effect Y. “Indirectly” in the context of a linkage between two entities refers to linkage in which the two entities do not contact one another, but are physically connected through one or more molecules or compounds which collectively contact both entities.

“Sequence identity” refers to the proportion of base matches between two nucleic acid sequences or the proportion of amino acid matches between two amino acid sequences. When sequence identity is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonuleotides as probes, the sequence identity between the target nucleic acid and the oligonucleotide sequence is preferably not less than 10 target base matches out of 20 (50% identity) and more preferably not less than about 60% identity, 70% identity, 80% identity or 90% identity, and most preferably not less than 95% identity.

“Selectively hybridize” refers to detectably and specifically bind. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence identity between the polynucleotides, oligonucleotides, and fragments thereof and a nucleic acid sequence of interest will be at least 30%, and more typically and preferably of at least 40%, 50%, 60%, 70%, 80% or 90%.

Hybridization and washing conditions are typically performed at high stringency according to conventional hybridization procedures. Positive clones are isolated and sequenced. For example, a full length polynucleotide sequence can be labeled and used as a hybridization probe to isolate genomic clones from an appropriate target library as they are known in the art. Typical hybridization conditions and methods for screening plaque lifts and other purposes are known in the art (Benton and Davis, Science 196:180 (1978); Sambrook et al., supra, (1989)).

Two amino acid sequences share identity if there is a partial or complete identity between their sequences. For example, 85% identity means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) share identity, as this term is used herein, if they have an alignment score of at least 5 (in standard deviation units) using the program ALIGN with the mutation data matrix and a gap penalty of 6 or greater (Dayhoff, in Atlas of Protein Sequence and Structure, National Biomedical Research Foundation, volume 5, pp. 101-110 (1972) and Supplement 2, pp. 1-10).

“Corresponds to” refers to a polynucleotide sequence that shares identity (for example is identical) to all or a portion of a reference polynucleotide sequence, or that a polypeptide sequence is identical to all or a portion of a reference polypeptide sequence. In contradistinction, the term “complementary to” is used herein to mean that the complementary sequence is homologous to or will base pair with all or a portion of a reference polynucleotide sequence. For illustration, the nucleotide sequence 5′-TATAC-3′ corresponds to a reference sequence 5′-TATAC-3′ and is complementary to a reference sequence 5′-GTATA-3′.

The following terms are used to describe the sequence relationships between two or more polynucleotides: “reference sequence,” “comparison window,” “sequence identity,” “percentage of sequence identity,” and “substantial identity.” A reference sequence is a defined sequence used as a basis for a sequence comparison; a reference sequence can be a subset of a larger sequence, for example, as a segment of a full length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (for example a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A comparison window, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window can comprise additions and deletions (for example, gaps) of 20 percent or less as compared to the reference sequence (which would not comprise additions or deletions) for optimal alignment of the two sequences. Optimal alignment of sequences for aligning a comparison window can be conducted by the local identity algorithm (Smith and Waterman, Adv. Appl. Math., 2:482 (1981)), by the identity alignment algorithm (Needleman and Wunsch, J. Mol. Bio., 48:443 (1970)), by the search for similarity method (Pearson and Lipman, Proc. Natl. Acid. Sci. U.S.A. 85:2444 (1988)), by the computerized implementations of these algorithms such as GAP, BESTFIT, FASTA and TFASTA (Wisconsin Genetics Software Page Release 7.0, Genetics Computer Group, Madison, Wis.), or by inspection. Preferably, the best alignment (for example, the result having the highest percentage of identity over the comparison window) generated by the various methods is selected.

“Complete sequence identity” means that two polynucleotide sequences are identical (for example, on a nucleotide-by-nucleotide basis) over the window of comparison.

“Percentage of sequence identity” is calculated by comparing two optimally aligned sequences over the window of comparison, determining the number of positions at which the identical nucleic acid base occurs in both sequences to yield the number of matched positions, dividing the number of matched positions by the total number of positions in the window of comparison (for example, the window size), and multiplying the result by 100 to yield the percentage of sequence identity.

“Substantial identity” as used herein denotes a characteristic of a polynucleotide sequence, wherein the polynucleotide comprises a sequence that has at least 30 percent sequence identity, preferably at least 50 to 60 percent sequence identity, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or addition which total 20 percent or less of the reference sequence over the window of comparison.

“Substantial identity” as applied to polypeptides herein means that two peptide sequences, when optimally aligned, such as by the programs GAP or BESTFIT using default gap weights, share at least 30 percent sequence identity, preferably at least 40 percent sequence identity, and more preferably at least 50 percent sequence identity, and most preferably at least 60 percent sequence identity. Preferably, residue positions that are not identical differ by conservative amino acid substitutions.

“Degenerate nucleic acid sequences” refers to nucleic acid sequences that include one or more degenerate codons. Degenerate nucleic acid sequences may use any sequence of nucleobases that encode the same sequence of amino acids as the reference sequence. For example, where the reference sequence comprises the sequence 5′-T-C-T-3′ encoding serine, a degenerate nucleic acid sequence may substitute 5′-T-C-T-3′, 5′-T-C-C-3′, 5′-T-C-A-3′, 5′-T-C-G-3′, 5′-A-G-T-3′, or 5′-A-G-C-3′. Examples of degenerate sequence codes includes but is not limited to the following (Table I and Table II).

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having acidic side chains is glutamic acid and aspartic acid; a group of amino acids having amino-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

TABLE I
Nucleotide Symbols

| Symbol | Meaning |
|---|---|
| A | A (adenine) |
| G | G (guanine) |
| C | C (cytosine) |
| T | T (thymine) |
| R | A or G (purine) |
| Y | T or C (pyrimidine) |
| M | A or C |
| K | G or T |
| S | G or C |
| W | A or T |
| B | G or C or T |
| D | A or G or T |
| H | A or C or T |
| V | A or G or C |
| N | A or G or C or T |

TABLE II
Degenerate Codons

| Amino | One-Letter | | Degenerate |
|---|---|---|---|
| Acid | Code | Codons | Codon |
| Cys | C | TGC TGT | TGY |
| Ser | S | AGC AGT TCA TCC TCG TCT | WSN |
| Thr | T | ACA ACC ACG ACT | CAN |
| Pro | P | CCA CCC CCG CCT | CCN |
| Ala | A | GCA GCC GCG GCT | GCN |
| Gly | 0 | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATO |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | | TAA TAG TGA | TRR |

“Modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

“Modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonists, partial agonists, antagonists, partial antagonists, antineoplastic agents, cytotoxic agents, inhibitors of neoplastic transformation or cell proliferation, cell proliferation promoting agents, antiviral agents, antimicrobial agents, antibacterial agents, antibiotics, and the like) by inclusion in assays described herein. The activity of a modulator may be known, unknown or partially known.

“Label” or “labeled” refers to incorporation of a detectable marker, for example by incorporation of a radiolabled compound or attachment to a polypeptide of moieties such as biotin that can be detected by the binding of a second moiety, such as marked avidin. Various methods of labeling polypeptides, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, beta-lactamase, horseradish peroxidase, alkaline phosphatase, luciferase; radioisotopes (such as ³H, ¹⁴C, ³⁵S, ¹²⁵I, ³²P or ¹³¹I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are beta-lactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued Sep. 8, 1998).

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods wherein the composition consists essentially of a single macromolecular species or activity. The inventors recognize that an activity may be caused, directly or indirectly, by a single species or a plurality of species within a composition, particularly with extracts.

A “bioactive derivative” refers to a modification of a bioactive compound or bioactivity that retains at least one characteristic activity of the parent compound.

A “bioactive precursor” refers to a precursor of a bioactive compound or bioactivity that exhibits at least one characteristic activity of the resulting bioactive compound or bioactivity.

A “patient” or “subject” refers a whole organism in need of or subjected to treatment, such as a farm animal, companion animal or human. An animal refers to any non-human animal.

An “avian species” includes all members of that classification, including domesticated members thereof, such as geese, chickens, ducks, turkeys, and quails.

A “gene disrupting sequence” is a nucleotide sequence that when inserted into a gene locus prevents expression of a gene. A gene disrupting sequence can prevent expression of a gene by preventing transcription of a gene, preventing appropriate splicing of a gene, or preventing appropriate translation of a gene. A gene disrupting sequence can be inserted into the coding region of a gene, into one or more introns of a gene, or into the 5′ or 3′ noncoding sequences of gene, or any combination thereof. A gene disrupting sequence can be a coding or noncoding sequence, for example, it can be a nucleotide sequence encoding a marker gene, or it can be sequences encoding stop codons, or it can be sequences that do not code for proteins.

Other technical terms used herein have their ordinary meaning in the art that they are used, as exemplified by a variety of technical dictionaries, such as the McGraw-Hill Dictionary of Chemical Terms and the Stedman's Medical Dictionary.

Introduction

The present invention recognizes that transgenic avian species, including chickens, can be engineered such that they can produce fully human antibodies, or chimeric human-avian antibodies, or humanized avian antibodies.

As a non-limiting introduction to the breadth of the present invention, the present invention includes several general and useful aspects, including:

1. methods of making avian cells and transgenic avians that:

a) lack endogenous heavy and light chain immunoglobulins, and

b) have exogenous immunoglobulin loci, or portions thereof; and

2. methods of making avian cells and transgenic avians for the generation of exogenous or chimeric antibodies.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

Aspect I: Methods of Making Transgenic Avian Cells with Deleted or Inactivated Immunoglobulin Heavy and Light Chain Loci

The present invention includes methods of making transgenic avian cells lacking functional endogenous immunoglobulin heavy and light chain loci, or portions thereof. Cells of the present invention can have at least one exogenous immunoglobulin locus, or at least one portion thereof. The cells of the present invention can be of any avian species, such as ducks, geese, turkeys, and quails, but are preferably chicken cells. In the following text, where chicken is used as an illustrative example, reference to all members of the avian species is intended and incorporated therein. The avian cells of the invention can be either primary cells or transformed cell lines, and may include any cell type, including for example, osteoblasts, osteoclasts, epithelial cells, endothelial cells, T-lymphocytes, neurons, glial cells, ganglion cells, retinal cells, liver cells, bone marrow cells, fibroblasts, keratinocytes, and myoblast (muscle) cells, but are preferably B-lymphocytes, embryonic stem (ES) cells, zygote (blastodermal) cells, sperm cells, or primordial germ cells.

The present invention includes the generation of genomic DNA deletions or gene disruptions in avian cells. The method of the invention provides the use of a replacement-type targeting construct to delete fragments of genomic DNA by gene targeting. Methods of creating non-human transgenic mammals using gene targeting are described in U.S. Pat. No. 5,998,209 issued Dec. 7, 1999 to Jakobovits, et al. and U.S. Pat. No. 6,066,778 issued May 23, 2000 to Ginsburg et al., both herein incorportated by reference. Methods for generating non-human transgenic mammals lacking a functional endogenous immunoglobulin locus and carrying a functional exogenous, preferably human, immunoglobulin locus are described in U.S. Pat. No. 5,939,598 issued Aug. 17, 1999 to Kucherlapati et al.; U.S. Pat. No. 6,114,598 issued Sep. 5, 2000 to Kucherlapati et al.; and U.S. Pat. No. 6,162,963 issued Dec. 19, 2000 to Kucherlapati et al., and PCT WO 94/02602, all herein incorporated by reference. The replacement targeting construct, which can contain a selectable marker, is constructed to contain two regions of sequences which are homologous to the 5′ and 3′ flanking sequences of the targeted locus. After transfection of the targeting construct into the desired cell line, gene targeted-mediated deletions may be identified by selection and further characterized by PCR, Southern blot analysis and/or pulsed field gel electrophoresis (PFGE).

The cells and transgenic avians which contain the genomic deletions may be used to study gene structure and function or biochemical processes such as, for example, protein production or inhibition. In addition, the transgenic avians may be used as a source of cells, organs, or tissues, or to provide model systems for human disease, such as for example, immune system disorders, or diseases such as Type I diabetes and multiple sclerosis, that may have an autoimmune component.

The transgenic avian cells may also be used to produce transgenic avians or avian cell lines producing chimeric or xenogeneic, preferably human, antibodies or modified antibodies. Genomic deletions or gene disruptions are created in the endogenous immunoglobulin loci in avian cells, and concurrently or in separate steps, the human heavy and light chain immunoglobulin gene complexes are introduced into the avian genome. This is accomplished by reconstructing the human heavy and light chain immunoglobulin genes, or portions thereof, in an appropriate eukaryotic or prokaryotic microorganism and introducing the resulting DNA fragments into avian cells, such as, but not limited to, cells that will become incorporated into the germ line of an avian.

Transgenic avians lacking functional immunoglobulin loci, or portions thereof, and having exogenous immunoglobulin loci, or portions thereof, can be immunized against an antigen of interest, and screened for production of antibodies that bind to the antigen of interest. Transgenic avians producing antibodies that bind to an antigen of interest can be used as a source of antibody that can be purified from eggs or from serum. Transgenic avians of the present invention producing antibodies that bind to an antigen of interest can also be used for the isolation of B-cells that can be immortalized, screened for the production of antibodies that bind with the antigen of interest, and grown in culture for the production of antibodies. Transgenic avians of the present invention producing antibodies that bind to an antigen of interest can also be used for the isolation of B-lymphocytes that can be used as a source of mRNA for cloning cDNAs that can encode human immunoglobulin light chains and/or immunoglobulin heavy chains. B-lymphocytes can be isolated from the bursa or spleen, or from the bone marrow, peripheral blood, gland of Harder, or intestinal lining of an avian. The sequences of immunoglobulin-encoding cDNAs can be optionally be altered using mutagenesis techniques and tested for enhanced or novel properties using phage display technologies. cDNAs encoding immunoglobulins (including altered immunoglobulins), or portions thereof, with desirable properties that are obtained by the methods of the present invention can be introduced into any appropriate cell type, such as, but not limited to, prokaryotic cells, yeast cells, insect cells, avian cells, or mammalian, including human, cells. Cells transformed with such cDNAs can be used for the production of immunoglobulins or polypeptides comprising portions of immunoglobulins.

Targeting Constructs and Introduction of Targeting Constructs into Avians and Avian Cells

For inactivation of avian immunoglobulin heavy chain and light chain loci, for each targeting event (heavy chain gene targeting and light chain gene targeting) a deletion can be generated in a targeting construct. The deletion will be flanked by sequences homologous to the avian Ig locus in which the deletion is being generated. The deletion will preferably be greater than 1 kb and preferably, will be within the range of 1 kb to 1000 kb. The deletion will normally include at least a portion of the coding region including a portion of one or more exons, a portion of one or more introns, and may or may not include a portion of the flanking noncoding regions, particularly the 5′-non-coding region (transcriptional regulatory region). Thus, the homologous region may extend beyond the coding region into the 5′-noncoding region or alternatively into the 3′-non-coding region. The homologous sequence should include at least about 300 bp. In the alternative, a lesion or gene disrupting sequence can be inserted in a portion of the locus that disrupts gene expression at the locus. Any lesion or sequence in the target locus resulting in the prevention of expression of an immunoglobulin subunit of that locus may be employed. Thus, the lesion or gene disrupting sequence may be in a region comprising the enhancer, e.g., 5′ upstream or intron, in the V, J or C regions, and with the heavy chain, the opportunity exists in the D region, or combinations thereof. Preferably, a deletion in the light chain gene comprises the entire variable region, such that none of the V genes remain intact after targeting. This avoids any possibility of a remaining V region recombining with exogenous genes that may be introduced into the host, for example by mechanisms such as gene conversion that may operate in chickens (Reynaud et al. Cell 48: 379-388 (1987)). Thus, the important factor is that Ig germ line gene rearrangement is inhibited, or a functional message encoding the immunoglobulin subunit cannot be produced, either due to failure of transcription, failure of processing of the message, or the like.

The replacement targeting construct can comprise at least a portion of the endogenous gene(s) at the selected locus for the purpose of introducing a deletion or gene disrupting sequence into at least one, preferably both, copies of the endogenous gene(s), so as to prevent its expression. For example, in chicken, there is a single light chain locus and a single heavy chain locus. The invention provides the use of a replacement-type targeting construct to delete the chicken light gene or portions thereof, including the psi V lambda cluster, L V lambda 1, J, and C lambda elements of genomic DNA, by gene targeting. Similarly, the chicken heavy chain gene, or portions thereof, including the psi V_{H} cluster, L V_{H1}, D cluster, J_{H} and C mu can be deleted using the methods of the present invention. The replacement-targeting construct may contain flanking sequences that are homologous to the 5′ and 3′ flanking sequences of the target. Such sequences can be obtained from regions of the chicken heavy chain and light chain loci (Reynaud et al. Cell 40: 283-291 (1985); Davies et al., J. Immunol. Methods 186: 125-135 (1995)).

When the deletion or gene disrupting sequence is introduced into only one copy of the gene being inactivated, the cells having a single unmutated copy of the target gene are expanded and may be subjected to a second targeting step, where the deletion or gene disrupting sequence may be the same or different from the first deletion and may overlap at least a portion of the deletion or gene disrupting sequence originally introduced. In this second targeting step, a targeting construct with the same arms of homology, but containing a different selectable marker, for example the hygromycin resistance gene (hyg-r) may be used to produce a clone containing a homozygous deletion. The resulting transformants are screened by standard procedures such as the use of negative or positive selection markers, and the DNA of the cell may be further screened to ensure the absence of a wild-type target gene, by standard procedures such as Southern blotting.

Alternatively when cells are targeted and are used to generate avians which are heterozygous for the deletion, homozygosity for the deletion or gene disruption may be achieved by cross breeding the heterozygous avians. Where it is advantageous to use cultured cells having disrupted endogenous immunoglobulin loci, such cells can be isolated from the homozygous transgenic animals, and, if advantageous, can be immortalized for continuous growth in culture. Immortalization of B-lymphocytes isolated from chickens and their use in antibody production is described in U.S. Pat. No. 5,049,502 issued Sep. 17, 1991 to Humphries, U.S. Pat. No. 5,258,299 issued Nov. 2, 1993, also to Humphries, and U.S. Pat. No. 6,143,559 issued Nov. 7, 2000 to Michael et al,, all herein incorporated by reference.

Another means by which homozygous deletions can be created in avian cells without the use of a second targeting step involves homogenization of the gene targeting event, as described in PCT application PCT/US93/00926, herein incorporated in its entirety by reference. In this method, the targeting construct is introduced into a cell in a first targeting step, to create the desired genomic deletion. The cells are then screened for gene-targeted recombinants, and the recombinants are exposed to elevated levels of the selection agent for the marker gene, in order to select for cells which have multiple copies of the selective agent by methods other than amplification. The cells are then analyzed for homozygosity at the target locus.

DNA vectors may be employed which provide for the desired introduction of the targeting construct into the cell. The constructs may be modified to include functional entities other than the deletion targeting construct which may find use in the preparation of the construct, amplification, transfection of the host cell, integration of the construct into the host cell, and integration of additional sequences into the construct sequences when integrated into the host genome.

The replacement targeting construct may include a deletion at one site and an insertion at another site which includes a gene for a selectable marker. Of particular interest is a gene which provides a marker, e.g., antibiotic resistance such as neomycin resistance. The presence of the selectable marker gene inserted into the target gene establishes the integration of the target vector into the host genome. However, DNA analysis will be required in order to establish whether homologous or non-homologous recombination occurred. This can be determined by employing probes for the insert and then sequencing the 5′ and 3′ regions flanking the insert for the presence of DNA extending beyond the flanking regions of the construct or identifying the presence of a deletion, when such deletion is introduced. The selectable marker may be flanked by recombinase target site sequences, such as lox, att, or frt sequences, such that it can be excised by supplying an appropriate recombinase, for example, cre, int, or flp recombinase, after selection of the transgenic cells and conformation of the homologously inserted sequence. Methods for excision of introduced sequences in transgenic cells using the cre-lox recombinase system is described in U.S. Pat. No. 6,066,778 issued May 23, 2000 to Ginsburg et al.

Another method for detecting cells in which the target gene has been deleted and which is especially useful when targeting genes which encode MHC Class I or II antigens, or immunoglobulin regions, involves the use of targeting constructs and an ELISA-based detection system, permitting the rapid detection of numerous independently targeted clones. In this method a site for homologous recombination is designed to create a recombinant fusion protein driven by a strong enhancer/promoter, for example the cytomegalovirus enhancer, fused to the domain of a protein containing an epitope, such as CD4. The epitope can be detected by a ligand to which it binds, for example an antibody, where the recombinant fusion protein is secreted by a correctly targeted cell and is then detected using an ELISA-based system employing antibodies that recognize the secreted fusion protein. In this method, the 5′ end of the recombinant locus is derived from the targeting construct, while the 3′ end of the locus is derived from the target gene. Because the entire 5′ end is controlled experimentally, both the recombinant fusion protein's expression level and ultimate transport fate can be directed. Media is screened to detect the fusion protein in an ELISA which traps proteins containing a beta₂-microglobulin epitope and detects proteins containing a CD4 epitope. In addition to a CD4 epitope, other peptides that contain an epitope recognized by a ligand, such as an antibody that binds to the epitope, may be used in the fusion protein.

In one preferred embodiment, at least a portion of the lesion is introduced into the J region of the immunoglobulin subunit locus, but this is not a requirement of the present invention. Preferably, the J region in whole or substantial part, usually at least about 75% of the locus, preferably at least about 90% of the locus, is deleted. Preferably, a deletion in the light chain gene comprises the entire variable region, such that none of the V genes remain intact after targeting, but this is not a requirement of the present invention. Deletion of the entire variable region avoids any possibility of a remaining V region recombining with exogenous genes that may be introduced into the host, for example by mechanisms such as gene conversion that may operate in chickens (Reynaud et al. Cell 48: 379-388 (1987)). Thus, one preferably produces a construct which lacks a functional J region and the entire V region of an immunoglobulin locus, and can comprise sequences adjacent to and upstream and/or downstream from V region, and can comprise sequences adjacent to and upstream and/or downstream from the J region. The insertion may be 50 bp or more, where such insertion of a gene disrupting sequence results in disruption of formation of a functional mRNA. The lesion between the two flanking sequences defining the homologous region can extend beyond the V and/or J regions, for example into or beyond the variable region and/or into the constant region.

Preferably, a marker gene is used to replace the V and/or J region. Various markers may be employed, particularly those which allow for positive selection. Of particular interest is the use of G418 resistance, resulting from expression of the gene for neomycin phosphotransferase.

Upstream and/or downstream from the target gene construct may be a gene which provides for identification of whether a double crossover has occurred. For this purpose, the Herpes simplex virus thymidine kinase gene may be employed, since cells expressing the thymidine kinase gene may be killed by the use of nucleoside analogs such as acyclovir or gancyclovir, by their cytotoxic effects on cells that contain a functional HSV-tk gene. The absence of sensitivity to these nucleoside analogs indicates the absence of the HSV-thymidine kinase gene and, therefore, where homologous recombination has occurred, that a double crossover has also occurred.

Where a selectable marker gene is involved, as an insert, and/or flanking gene, depending upon the nature of the gene, it may be from a host where the transcriptional initiation region (promoter) is not recognized by the transcriptional machinery of the avian host cell. In this case, a different transcriptional initiation region (promoter) will be required. This region may be constitutive or inducible. A wide variety of transcriptional initiation regions have been isolated and used with different genes. Of particular interest is the promoter region of rous sarcoma virus. In addition to the promoter, the wild type enhancer may be present or an enhancer from a different gene may be joined to the promoter region.

While the presence of the marker gene in the genome will indicate that integration has occurred, it is preferable to further determine whether homologous integration has occurred. This can be achieved in a number of ways. For the most part, DNA analysis will be employed to establish the location of the integration. By employing probes for the insert and then sequencing the 5′ and 3′ regions flanking the insert for the presence of the target locus extending beyond the flanking region of the construct or identifying the presence of a deletion, when such deletion has been introduced, the desired integration may be established.

The polymerase chain reaction (PCR) can be used with advantage in detecting the presence of homologous recombination. Probes may be used which are complementary to a sequence within the construct and complementary to a sequence outside the construct and at the target locus. In this way, one can only obtain DNA chains having both the primers present in the complementary chains if homologous recombination has occurred. By demonstrating the presence of the PCR product for the expected size using such primers, the occurrence of homologous recombination is supported.

In constructing the subject constructs for homologous recombination, a replication system for procaryotes, particularly E. coli, may be included, for preparing the construct, cloning after each manipulation, analysis, such as restriction mapping or sequencing, or expansion and isolation of the desired sequence. Where the construct is large, generally exceeding about 50 kbp, usually exceeding 100 kbp, and usually not more than about 1000 kbp, a yeast artificial chromosome (YAC) may be used for cloning of the construct. When necessary, a different selectable marker may be employed for detecting bacterial or yeast transformations.

Once a construct has been prepared and, optionally, any undesirable sequences removed, e.g., procaryotic sequences, the construct may now be introduced into the target cell. Any convenient technique for introducing the DNA into the target cells may be employed. Techniques which may be used to introduce the replacement targeting construct into the avian cells include calcium phosphate/DNA coprecipitates, microinjection of DNA into the nucleus, electroporation, bacterial or yeast protoplast fusion with intact cells, transfection, particle gun bombardment, lipofection or the like. Where avian embryonic stem cells are used as the recipient cells, the DNA can be targeted to the cells using liposomes (Pain et al. Cells Tissues Organs 165: 212-219 (1999)). Where avian zygotes are used, the construct can be microinjected into the cytoplasm of the germinal disc (Love et al. Bio/Technology 12: 60-63 (1994). The DNA may be single or double stranded, linear or circular, relaxed or supercoiled DNA. After transformation or transfection of the target cells, target cells may be selected by means of positive and/or negative markers, as previously indicated, neomycin resistance and acyclovir or gancyclovir resistance. Those cells which show the desired phenotype may then be further analyzed by restriction analysis, electrophoresis, Southern analysis, PCR, or the like. By identifying fragments which show the presence of the lesion(s) at the target locus, one can identify cells in which homologous recombination has occurred to inactivate a copy of the target locus.

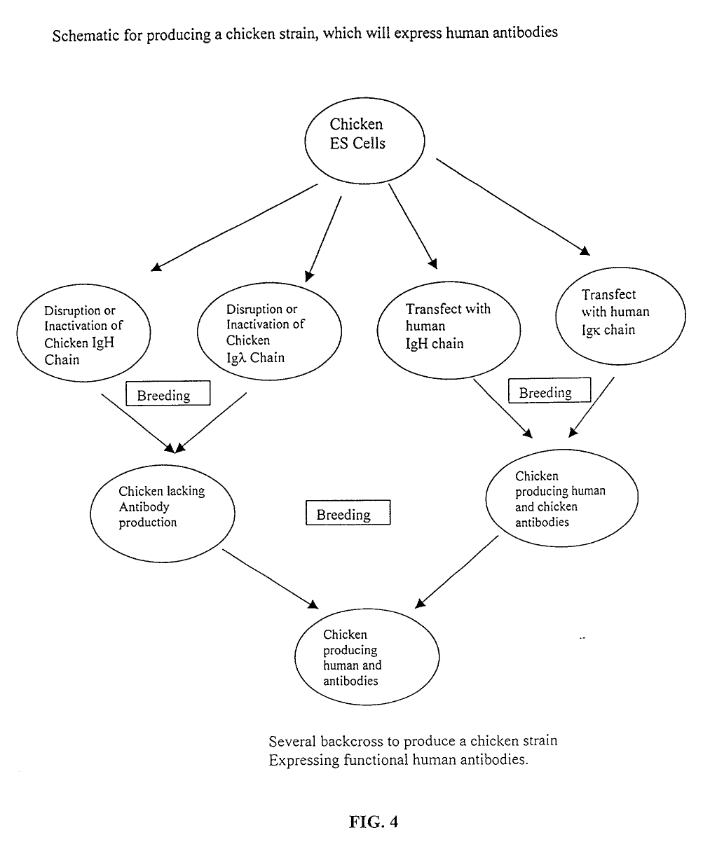

The above described process may be performed first with a heavy chain locus in an embryonic stem cell and then maturation of the cells to provide a mature fertile host. Then by breeding of the heterozygous hosts, a homozygous host may be obtained or embryonic stem cells may be isolated and transformed to inactivate the second Ig_{H} locus, and the process repeated until all the desired loci have been inactivated. Alternatively, the light chain locus may be the first. At any stage, the human loci may be introduced. A breeding strategy to generate transgenic chickens lacking functional endogenous immunoglobulin loci and having human immunoglobulin loci is depicted in FIG. 4.

In one strategy, as individual steps, the avian heavy and light chain immunoglobulin gene complexes are rendered non-functional and in a separate step the corresponding human genes are introduced into avian cells. Inactivation of the endogenous avian immunoglobulin loci is achieved by targeted disruption of the appropriate loci by homologous recombination in avian cells. Human heavy and light chain genes are reconstructed in an appropriate eukaryotic or prokaryotic microorganism and the resulting DNA fragments can be introduced into the avian cells. The human light and heavy chain loci can be provided in one or more yeast artificial chromosomes (YACs). The entire Ig_{H} hu locus can be contained within one or a few yeast artificial chromosome (YAC) clones. The same is true for the Ig light chain loci. Subsequent introduction of the appropriate heavy chain or light chain YAC clones into recipient yeast allows for the reconstitution of intact germ line Ig loci by homologous recombination between overlapping regions of homology. In this manner, the isolation of DNA fragments encoding the human Ig chain can be achieved. In another strategy, the human light and heavy chain loci are provided in targeting vectors that integrate into the avian light and heavy chain loci and thereby inactivate the endogenous loci.

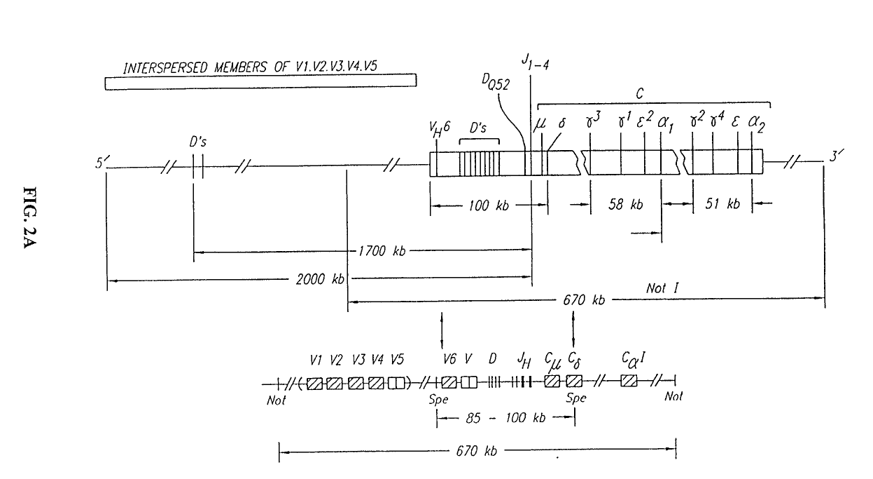

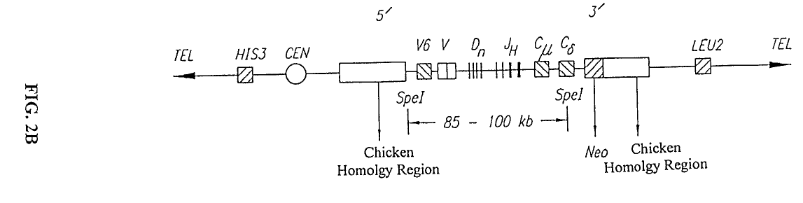

In order to obtain a broad spectrum of high affinity human antibodies from a transgenic avian, it is not necessary that one include the entire human V regions. Various V region gene families are interspersed within the V region cluster. Thus, by obtaining a subset of the known V region genes of the human heavy and light chain Ig loci (Berman et al., EMBO J. (1988) 7: 727-738) rather than the entire complement of V regions, the transgenic host may be immunized and be capable of mounting a strong immune response and provide high affinity antibodies. In this manner, relatively small DNA fragments of the chromosome may be employed, for example, a reported 670 kb fragment of the Ig Hu locus is shown in FIG. 2A. This NotI-NotI restriction fragment would serve to provide a variety of V regions, which will provide increased diversity by recombining with the various D and J regions and undergoing somatic mutation.

These strategies are based on the known organization of the immunoglobulin chain loci in a number of animals, since the organization, relative location of exons encoding individual domains, and location of splice sites and transcriptional elements, is understood to varying degrees. In the human, the immunoglobulin heavy chain locus (Ig_{H}) is located on chromosome 14. In the 5′-3′ direction of transcription, the locus comprises a large cluster of variable region genes (V_{H}), the diversity (D) region genes, followed by the joining (J_{H}) region genes and the constant (C_{H}) gene cluster. The size of the locus is estimated to be about 2,500 kilobases (kb). During B-cell development, discontinuous gene segments from the germ line Ig_{H} locus are juxtaposed by means of a physical rearrangement of the DNA. In order for a functional heavy chain Ig polypeptide to be produced, three discontinuous DNA segments, from the V_{H}, D, and J_{H} regions must be joined in a specific sequential fashion; V_{H} to DJ_{H}, generating the functional unit V_{H} DJ_{H}. Once a V_{H} DJ_{H} has been formed, specific heavy chains are produced following transcription of the Ig locus, utilizing as a template the specific V_{H} DJ_{H} C_{H} unit comprising exons and introns. There are two loci for Ig light chains (Ig_{L}), the kappa locus on human chromosome 2 and the lambda. locus on human chromosome 22. The structure of the Ig_{L} loci is similar to that of the Ig_{H} locus, except that the D region is not present. Following Ig_{H} rearrangement, rearrangement of a light chain locus is similarly accomplished by V_{L} and J_{L} joining of the kappa or lambda chain. The sizes of the lambda and kappa loci are each approximately 1000 kb. Expression of rearranged Ig heavy chain and an Ig kappa or Ig lambda light chain in a particular B-cell allows for the generation of antibody molecules.

In order to isolate, clone and transfer the Ig_{H} hu locus, a yeast artificial chromosome may be employed. A preferred target construct is a YAC containing human heavy chain complex containing V_{H}, D_{H}, J_{H}, C mu and C delta, and a selection marker such as the G418 or neomycin resistance gene. Similarly, for targeted disruption of light chain in an avian, the target construct may contain variable region genes, J regions, and kappa or lambda constant region genes, and a second selection marker, which may be thymidine kinase (tk) or DHFR. Both of these vectors will contain 5′ and 3′ flanking sequence of avian heavy and light chain gene complex flanking the human heavy and light chain genes, respectively. This would allow replacement of the human genes at the analogous position in an avian.

It is preferable, although not necessary, to target the human heavy and light chain loci to the disrupted avian heavy and light chain chromosomal loci. Where transgenic birds are to be generated, this arrangement allows for simplified breeding to achieve birds that simultaneously lack an endogenous immunoglobulin locus and possess an exogenous immunoglobulin locus. In addition, the human locus will be placed substantially in the same region as the analogous host locus, so that any regulation associated with the position of the locus will be substantially the same for the human immunoglobulin locus. For example, by isolating the entire V_{H} gene locus (including V, D, and J sequences), or portion thereof, and flanking the human locus with sequences from the corresponding avian locus, preferably sequences separated by at least about 1 kbp, in the host locus, preferably at least about 5 kbp in the host locus, one may insert the human fragment into this region in one or more recombinational events, substituting the human immunoglobulin locus for the variable region of the host immunoglobulin locus. In this manner, one may disrupt the ability of the host to produce an endogenous immunoglobulin subunit, while allowing for the promoter of the human immunoglobulin locus to be activated by the host enhancer and regulated by the regulatory system of the host.

The construct carrying the exogenous immunoglobulin locus can therefore contain sequences of the endogenous avian immunoglobulin locus in order to promote homologous recombination of the exogenous immunoglobulin locus into the endogenous immunoglobulin locus. In another strategy, the gene disruption construct employed in inactivating the endogenous immunoglobulin loci can also transfer specific integration sequences to the disrupted locus. Sequences such as “lox”, “att”, and “frt” that allow highly efficient targeted integration of gene sequences can be introduced to these loci, and transient expression of the corresponding cre, int, or FLP recombinase can provide for efficient recombination of the introduced human sequences into the endogenous disrupted loci. Integration of the human sequences can occur simultaneously with excision of selectable marker sequences introduced into the locus by the gene disruption construct. Methods of using sequence-specific recombinase target sites and corresponding recombinases to fuse sequences or insert sequences is described in U.S. Pat. No. 4,959,317 issued Sep. 25, 1998 to Sauer, et al., U.S. Pat. No. 5,851,808 issued Dec. 22, 1998 to Elledge et al., U.S. Pat. No. 5,998,144 issued Dec. 7, 1999 to Reff et al., and U.S. Pat. No. 6,066,778 issued May 23, 2000 to Ginsburg et al., all herein incorporated by reference.

In the alternative, it is possible to have integration of the exogenous immunoglobulin loci in other regions of the genome. In this instance, it can also be desirable to provide sequences in the genome, such as lox, att, or frt sites, as target sites for integration of the exogenous immunoglobulin loci to promote more efficient gene transfer. Such recombinase target sites can be introduced into the host genome using vectors introduced into avian cells by any adequate method, including, for example, spheroplast fusion, lipofection, electroporation, calcium phosphate-mediated DNA transfer, particle gun bombardment, retroviral infection, or direct microinjection. Of particular relevance in avians is the use of replication-defective retroviruses that can be used to infect cells in culture or injected into the developing embryo and provide a high frequency of chromosomal integration (see, for example, U.S. Pat. No. 5,162,215 issued Nov. 10, 1992 to Bosselman et al.). Such retroviral vectors can be used to provide recombination “acceptor”sites for the integration of exogenous immunoglobulin loci, as described in U.S. Pat. No. 5,998,144.

For the generation of transgenic avian cells the human DNA, preferably in a YAC vector, may be introduced into into avian cells by any adequate method, including, for example, spheroplast fusion, lipofection, electroporation, calcium phosphate-mediated DNA transfer, particle gun bombardment, retroviral infection, or direct microinjection. The integration may be random, homologous, recombinase-mediated, or retrovirally-mediated depending on the particular strategy to be employed. For the generation of transgenic birds, replication-defective retroviruses can also be injected into the developing embryo (see, for example, U.S. Pat. No. 5,162,215 issued Nov. 10, 1992 to Bosselman et al.). The exogenous Ig locus can be introduced into avian cells or avian animals that do not have disrupted endogenous Ig loci, and the enodogenous Ig loci can be disrupted in later steps.

Alternatively, transgenic birds carrying exogenous Ig loci and lacking endogenous Ig loci can be generated by selective breeding. A breeding strategy to generate transgenic chickens lacking functional endogenous heavy and light chain immunoglobulin loci having human immunoglobulin heavy and light chain loci is depicted in FIG. 4. For example, the modified avian cells with a disrupted immunoglobulin locus, for example, a disrupted heavy chain locus, can be used to generate transgenic avians that transmit the disrupted heavy chain locus through the germ line and modified avian cells with a disrupted light chain locus can be used to generate transgenic avians that transmit the disrupted light chain locus through the germ line. Mating of avians that have disrupted heavy chain loci with avians that have disrupted light chain loci will produce progeny that lack both heavy and light chain immunoglobulins. Correspondingly, transgenic avians having a human immunoglobulin light chain locus can be mated with trangenic avians that have human immunoglobulin heavy chain locus to produce progeny that produce human antibodies. The mating of avian strains with human immunoglobulin loci to strains with inactivated avian loci will yield animals whose antibody production is purely human.

Once the human loci have been introduced into the host genome, either by homologous recombination, the use of lox, att, or frt sequences, or random integration, and host animals have been produced with the endogenous immunoglobulin loci inactivated by appropriate breeding of the various transgenic or mutated animals, one can produce a host which lacks the native capability to produce endogenous immunoglobulin subunits, but has the capacity to produce human immunoglobulins with at least a significant portion of the human repertoire.

The functional inactivation of the two copies of each of the two host Ig loci, where the host contains the human Ig_{H} and the human Ig kappa and/or lambda loci, would allow for the production of purely human antibody molecules without the production of host or host/human chimeric antibodies. Such a host strain, by immunization with specific antigens, would respond by the production of avian B-cells producing specific human antibodies, which B-cells could be immortalized in any manner for the continuous stable production of human monoclonal antibodies.

The subject methodology and strategies need not be limited to producing complete immunoglobulins, but provides the opportunity to provide for regions joined to a portion of the constant region, e.g., CH_{H1}, CH_{H2}, CH_{H3}, or CH_{H4}, or combination thereof. Alternatively, one or more of the exons of the C_{H} and C light chain regions may be replaced or joined to a sequence encoding a different protein, such as an enzyme, e.g., plasminogen activator, superoxide dismutase, etc.; toxin A chain, e.g., ricin, abrin, diphtheria toxin, etc.; growth factors; cytotoxic agent, e.g., TNF, or an reporter protein, such as green fluorescent protein, beta galactosidease, alkaline phosphatase, or a specific binding protein or epitope such as glutathione-S-transferase, streptavidin, a series of histidine residues, or the like. See, for example, WO 89/07142; WO 89/09344; and WO 88/03559. By inserting the protein of interest into a constant region exon and providing for splicing of the variable region to the modified constant region exon, the resulting binding protein may have a different C-terminal region from the immunoglobulin. By providing for a stop sequence with the inserted gene, the protein product will have the inserted protein as the C-terminal region. If desired, the constant region may be entirely substituted by the other protein, by providing for a construct with the appropriate splice sites for joining the variable region to the other protein. Proteins useful in this regard include those listed above.

The antibodies or antibody analog producing B-cells from the transgenic host may be immortalized e.g., by transfection with oncogenes. Oncogenes may be transmitted by a retrovirus such as reticuloendotheliosis virus (see for example, U.S. Pat. No. 5,258,299, U.S. Pat. No. 5,049,502, and U.S. Pat. No. 5,028,540, all herein incorporated by reference), or the oncogene can be introduced independently of a retrovirus (such as in the context of a plasmid or other vector) and can be introduced by electroporation or other transfection techniques. It is also possible to immortalize avian B-lymphocytes by fusing them with immortalized cell lines, preferably cell lines of the same species as the B-lymphocytes. For example, chicken B-lymphocytes can be fused with R24H4, a hybrid TK-chicken lymphoblastoid cell line (Nishinaka et al. (1989) (1991) or DT40 (Baba et al. 1985). Methods of inducing cell fusion are known in the art and can include the use of polymers such as PEG or electrical current. These immortalized cells may then be grown in continuous culture or transplanted into the another avian to expand the cells, which can be re-isolated from the spleen, bursa, bone marrow, liver, intestinal lining, gland or Harder, or peripheral blood of the second bird and screened for production of antibodies with activity against the desired antigen.

The subject invention provides for the production of polyclonal human antibodies from avian serum or eggs (see, for example, Mohammed et al. Immunotechnology 4: 115-125 (1998)) or human monoclonal antibodies or antibody analogs. Where the avian host has been immunized with an immunogen, the resulting human antibodies may be isolated from other proteins by using an affinity column, having an Fc binding moiety, such as protein A, or the like.

In order to provide for the production of human antibodies in a xenogeneic host, it is necessary that the host be competent to provide the necessary enzymes and other factors involved with the production of antibodies, while lacking competent endogenous genes for the expression of heavy and light subunits of immunoglobulins. Thus, those enzymes and other factors associated with germ line rearrangement, splicing, somatic mutation, glycosylation, and the like, will be functional in the xenogeneic host. Although gene rearrangement is not a key event in Ig diversity in chicken, which is a preferred avian of the present invention, chicken B cells express proteins that are responsible for Ig gene rearrangement. Heptamer sequences specific for the rearrangement process exist in two locations within the V lambda 1 gene and also in the V_{H1} and half of the D elements. The RAG-2 gene which is required for V(D)J DNA recombination at loci for Ig and T cell receptor genes is highly expressed in chicken bursa. The gene encoding the other protein used in immunoglobulin rearrangement in mammals, RAG-1, is also expressed in chicken bursa. Chicken Ig genes in transgenic mouse undergo gene rearrangements suggesting that evolutionarily conserved enzymes are used for Ig gene rearrangement.

Methods for Generating Transgenic Avians

When genetic loci of zygote cells from an avian host, have been targeted and/or transfected with exogenous immunoglobulin sequences, it may be desirable to use such cells to generate transgenic animals. For such a procedure, following the introduction of the targeting construct into the embryonic stem (ES) cells, the cells may be plated onto a feeder layer in an appropriate medium, for example, DMEM supplemented with growth factors and cytokines, fetal bovine serum and antibiotics (Pain et al. 1996). The embryonic stem cells may have a single targeted locus (heterozygotic) or both loci targeted (homozygotic). Cells containing the construct may be detected by employing a selective medium and after sufficient time for colonies to grow, colonies may be picked and analyzed for the occurrence of gene targeting. As described previously, PCR may be used, with primers within and outside the construct sequence, or Southern blot analysis or PFGE, but at the target locus. Those colonies which show gene targeting may then be used for injection into avian embryos. The ES cells can then be trypsinized and the modified cells can be injected through a an opening made in the side of the egg as described in U.S. Pat. No. 5,162,215. After sealing the eggs, the eggs can be incubated at 37 degrees C. until hatching. Newly hatched avians can be tested for the presence of the target construct sequences, for example by removing a blood sample. After the avians have reached maturity, they are bred and their progeny are examined to determine whether the gene targeting sequences are transmitted through the germ line.

Chimeric avians are generated which are derived in part from the modified embryonic stem cells or zygote cells, capable of transmitting the genetic modifications through the germ line. Mating avian strains containing human immunoglobulin loci, or portions thereof, to strains with strains in which the avian immunoglobulin loci, or portions thereof, have been deleted generates avians which produce chimeric or purely human antibodies.

Transgenic avians can also be produced by other methods, some of which are discussed below. Among the avian cells suitable for transformation for generating transgenic animals are sperm cells, primordial germ cells, and zygote cells (including embryonic stem cells). Sperm cells can be transformed with DNA constructs by any suitable method, including electroporation, microparticle bombardment and lipofection (Gruenbaum et al. J. Cell. Biochem.15E, 194(1991); Rottman et al., J. Anim. Breed. Genet. 109: 64-70 (1992); Squires and Drake, Anim. Biotechnol. 4: 71-88 (1993). The sperm can be used for artificial insemination of avians. Progeny of the inseminated avians can be examined for the targeting sequence as described above.

Alternatively, primordial germ cells (Petitte et al. Poult. Sci. 76: 1084-92 (1997) can be isolated from avian eggs (Vick et al., Proc. R. Soc. London Ser. B 251: 179-182 (1993); Tajima et al., Theriogenology 40: 509-519 (1993)), transfected with targeting constructs by any appropriate method, and transferred into new embryos, where they can become incorporated into the developing gonads. Hatched avians and their progeny can be examined for the targeting sequence as described above.

In yet another approach, dispersed blastodermal cells isolated from eggs can be transfected by any appropriate means with a targeting construct or constructs containing exogenous immunoglobulin loci, or portions thereof, and injected into the subgerminal cavity of intact eggs (Carscience et al. Development 117: 669-75 (1993). Hatched avians and their progeny can be examined for the targeting sequence as described above.

One of the advantages of the avian system is that the zygote is highly accessible to the researcher as it develops external to the female organism. For example, eggs containing developing zygotes can be injected with DNA constructs (Bosselman, R. A. et al., Science 243:533-535 (1989), and described in U.S. Pat. No. 5,162,215 ), or DNA can be introduced into cells of developing zygotes that are cultured outside the egg ( Perry, Nature 331: 70-72 (1988), Love et al. Bio/Technol. 12: 60-63 (1994), and Naito et al. Mol. Reprod. Dev. 37: 167-171 (1994) ). This is particularly useful where retroviral constructs are used, such as in the introduction of relatively small gene segments or recombination target sites.

In accordance with the above procedures, an avian host can be produced which can be immunized to produce human antibodies or analogs specific for an immunogen. In this manner, the problems associated with obtaining human monoclonal antibodies are avoided, since avians can be immunized with immunogens which could not be used with a human host. Furthermore, one can provide for booster injections and adjuvants, which would not be permitted with a human host. The resulting B-cells may then be used for immortalization for the continuous production of the desired antibody.

The immortalized cells can also be used for isolation of the genes encoding the immunoglobulin or analog and the genes can optionally be subjected to mutation by in vitro mutagenesis or other mutagenizing technique. Phage display methodologies can be used to select for nucleic acid sequences encoding immunoglobulins, or portions thereof, with modified properties (Davies, et al., J. Immunol. Methods 186: 125-135 (1995); and see also U.S. Pat. Nos. 5,223,409, 5,846,533, and 5,824,520, all herein incorportated by reference). These mutagenized nucleic acid sequences may then be returned to the immortalized cells or to other cell lines to provide for a continuous avian cellular source of the desired antibodies. The subject invention provides for a convenient source of human antibodies, where the human antibodies are produced in analogous manner to the production of antibodies in a human host. The avian cells can conveniently provide for the activation and rearrangement of human DNA in avian cells for production of human antibodies.

In vitro Cultures of Avian Cells with Modified Immunoglobulin Loci

While the foregoing discussion provides methods for generation of human antibodies in transgenic avians, the invention also encompasses the use of methods of the present invention for disrupting endogenous immunoglobulin loci in cells that can be grown continuously in vitro.

Avian cells with disrupted endogenous loci can be used for the expression of exogenous antibodies, such as human antibodies. Cells that have disrupted endogenous immunoglobulin loci can be transfected with nucleic acids, such as, but not limited to, cDNAs, that encode exogenous proteins, such as human proteins. Preferred nucleic acids of this aspect of the invention are DNAs that encode immunoglobulin heavy chain genes and DNAs that encode immunoglobulin light chain genes. Such DNAs can be modified, such as to provide sequences that can improve expression in the genome, or to change the properties of an immunoblobulin encoded by the DNAs, such as, but not limited to, its binding properties.

For example, the genes encoding the immunoglobulin or analog can be subjected to mutation by in vitro mutagenesis or other mutagenizing technique, that can be combined with techniques such as phage display to select for antibodies with modified properties (Davies, et al., J. Immunol. Methods 186: 125-135 (1995); and see also U.S. Pat. Nos. 5,223,409, 5,846,533, and 5,824,520, all herein incorporated by reference). These mutagenized genes may then be returned to the immortalized cells or introduced into other cells lines to provide for a continuous cellular source of the desired antibodies. The subject invention provides for a convenient source of human antibodies, where the human antibodies are produced in analogous manner to the production of antibodies in a human host.

In this aspect, the subject methodology and strategies need not be limited to producing complete immunoglobulins, but provides the opportunity to provide for regions of exogenous immunoglobulin genes joined to a sequence encoding a different protein, such as an enzyme, for example, plasminogen activator, superoxide dismutase, etc.; toxin A chain, for example, ricin, abrin, diphtheria toxin, etc.; growth factors; cytotoxic agent, for example, TNF, or the like, or an reporter protein, such as green fluorescent protein, beta galactosidease, or alkaline phosphatase, or a specific binding protein or peptide such as glutathione-S-transferase, streptavidin, a series of histidine residues, or the like. See, for example, WO 89/07142; WO 89/09344; and WO 88/03559. If desired, all or a portion of the constant region of an exogenous immunoglobulin gene may be substituted by the other protein.

The avian cells which contain the genomic deletions may also be used to study gene structure and function or biochemical processes such as, for example, protein production or inhibition.

The present invention therefore includes methods of making transgenic avian cells lacking functional endogenous immunoglobulin heavy and light chain loci, or portions thereof. Cells of the present invention can have at least one exogenous immunoglobulin locus, or at least one portion thereof. The cells of the present invention can be of any avian species, such as but not limited to ducks, geese, turkeys, and quails, but are preferably chicken cells. The avian cells of the invention can be either primary cells or transformed cell lines, and may include any cell type, including for example, osteoblasts, osteoclasts, epithelial cells, endothelial cells, fibroblasts, T-lymphocytes, neurons, glial cells, ganglion cells, retinal cells, liver cells, bone marrow cells, fibroblasts, keratinocytes, and myoblast (muscle) cells, embryonic stem cells, zygote cells, sperm cells, or primordial germ cells, but are preferably B-lymphocytes or cell lines derived from B-lymphocytes, including hybrid cell lines derived from B-lymphocytes.

The present invention includes the generation of genomic DNA deletions or gene disruptions in avian cells. The method of the invention provides the use of a replacement-type targeting construct to delete fragments of genomic DNA by gene targeting. Methods of gene targeting are described in U.S. Pat. No. 5,998,209 issued Dec. 7, 1999 to Jakobovits, et al., and U.S. Pat. No. 6,066,778 issued May 23, 2000 to Ginsburg et al., both herein incorporated by reference. Methods for generating cells lacking a functional endogenous immunoglobulin locus and carrying a functional exogenous, preferably human, immunoglobulin locus are described in U.S. Pat. No. 5,939,598 issued Aug. 17, 1999 to Kucherlapati et al., and PCT WO 94/02602, both herein incorporated by reference. The replacement targeting construct, which can contain a selectable marker, is constructed to contain two regions of sequences which are homologous to the 5′ and 3′ flanking sequences of the targeted locus. After transfection of the targeting construct into the desired cell line, gene targeted-mediated deletions may be identified by selection and further characterized by PCR, Southern blot analysis and/or pulsed field gel electrophoresis (PFGE).

In a preferred aspect of the invention, genomic deletions or gene disruptions are created in the endogenous immunoglobulin loci in avian cells, and concurrently or in separate steps, human heavy and light chain immunoglobulin genes are introduced into the avian genome. This is accomplished by reconstructing a human heavy chain gene and/or a human light chain gene, or portions thereof, in an appropriate eukaryotic or prokaryotic microorganism and introducing the resulting DNA fragments into avian cells that lack expression of endogenous immunoglobulin heavy and light chains.

Aspect II: Methods of Generating Transgenic Avian Cells and Avians Producing Chimeric Immunoglobulins

The present invention includes methods of making transgenic avian cells lacking functional endogenous immunoglobulin heavy chain constant regions and endogenous immunoglobulin light chain constant regions, or portions thereof. Cells of the present invention can have at least one exogenous immunoglobulin constant region, or at least one portion thereof. The cells of the present invention can be of any avian species, such as but not limited to ducks, geese, turkeys, and quails, but are preferably chicken cells. In the following text, where chicken is used as an illustrative example, reference to all avian species is intended and incorporated herein. The avian cells of the invention can be either primary cells or transformed cell lines, and may include any cell type, including for example, osteoblasts, osteoclasts, epithelial cells, endothelial cells, fibroblasts, T-lymphocytes, neurons, glial cells, ganglion cells, retinal cells, liver cells, bone marrow cells, fibroblasts, keratinocytes, and myoblast (muscle) cells, but are preferably B-lymphocytes, embryonic stem (ES) cells, zygote (blastodermal) cells, sperm cells, or primordial germ cells.

The present invention includes the generation of genomic DNA deletions or gene disruptions in avian cells. The method of the invention provides the use of a replacement-type targeting construct to delete fragments of genomic DNA by gene targeting. Methods of creating non-human transgenic mammals using gene targeting are described in U.S. Pat. No. 5,998,209 issued Dec. 7, 1999 to Jakobovits, et al., and U.S. Pat. No. 6,066,778 issued May 23, 2000 to Ginsburg et al., both herein incorportated by reference. Methods for generating non-human transgenic mammals lacking a functional endogenous immunoglobulin locus and carrying a functional exogenous, preferably human, immunoglobulin locus are described in U.S. Pat. No. 5,939,598 issued Aug. 17, 1999 to Kucherlapati et al.; U.S. Pat. No. 6,114,598 issued Sep. 5, 2000 to Kucherlapati et al.; and U.S. Pat. No. 6,162,963 issued Dec. 19, 2000 to Kucherlapati et al., and PCT WO 94/02602, all herein incorporated by reference. The replacement targeting construct, which may contain a selectable marker, is constructed to contain two regions of sequences which are homologous to the 5′ and 3′ flanking sequences of the targeted locus. After transfection of the targeting construct into the desired cell line, gene targeted-mediated deletions may be identified by selection and further characterized by PCR, Southern blot analysis and/or pulsed field gel electrophoresis (PFGE).

The cells and transgenic avians which contain the genomic deletions may be used to study gene structure and function or biochemical processes such as, for example, protein production or inhibition. In addition, the transgenic avians may be used as a source of cells, organs, or tissues, or to provide model systems for human disease, such as for example, immune system disorders, or diseases such as Type I diabetes and multiple sclerosis, that may have an autoimmune component.

The transgenic avian cells may also be used to produce transgenic avians producing chimeric, preferably human-avian, antibodies or modified antibodies. Genomic deletions or gene disruptions are created in the constant regions of endogenous immunoglobulin loci in avian cells, and concurrently or in separate steps, the human heavy and light chain immunoglobulin gene constant regions are introduced into the avian genome. This is accomplished by reconstructing the human heavy and light chain immunoglobulin gene constant regions, or portions thereof, in an appropriate eukaryotic or prokaryotic microorganism and introducing the resulting DNA fragments into avian cells, such as cells that will become incorporated into the germ line of an avian.

Targeting Constructs and Introduction of Targeting Constructs into Avians and Avian Cells

For diruption of avian immunoglobulin heavy chain and light chain constant regions, for each targeting event (heavy chain gene constant region targeting and light chain constant region gene targeting) a deletion can be generated in a targeting construct. The deletion will be flanked by sequences homologous to the those bordering the constant region of the avian Ig locus in which the deletion is being generated. The deletion will preferably be greater than 0.5 kb and preferably, will be within the range of 0.5 kb to 10 kb. The deletion will normally include all of the constant region coding regions, and may or may not include a portion of the flanking noncoding regions. Thus, the homologous region may extend beyond the coding region into the 5′-noncoding region or alternatively into the 3′-non-coding region surrounding the constant region gene segments. The homologous sequence should include at least about 300 bp.

The replacement targeting construct will comprise at least a portion of the endogenous gene(s) at the selected locus for the purpose of introducing a deletion or gene disrupting sequence into at least one, preferably both, copies of the endogenous gene(s), so as to prevent its expression. In chicken, for example, there is a single light chain locus and a single heavy chain locus. The invention provides the use of a replacement-type targeting construct to delete the chicken light chain C lambda gene. Similarly, the C mu cluster of the chicken heavy chain gene can be deleted using the methods of the present invention. The replacement-targeting construct may contain flanking sequences that are homologous to the 5′ and 3′ flanking sequences of these targets.

When the deletion is introduced into only one copy of the gene being inactivated, the cells having a single unmutated copy of the target gene are expanded and may be subjected to a second targeting step, where the deletion may be the same or different from the first deletion and may overlap at least a portion of the deletion originally introduced. In this second targeting step, a targeting construct with the same arms of homology, but containing a different selectable marker, for example the hygromycin resistance gene (hyg-r) may be used to produce a clone containing a homozygous deletion. The resulting transformants are screened by standard procedures such as the use of negative or positive selection markers, and the DNA of the cell may be further screened to ensure the absence of a wild-type target gene, by standard procedures such as Southern blotting.

Alternatively when cells are targeted and are used to generate birds which are heterozygous for the deletion, homozygosity for the deletion or gene disruption may be achieved by cross breeding the heterozygous avians. Where it is advantageous to use cultured cells having mutated endogenous immunoglobulin loci, such cells can be isolated from the homozygous transgenic avians, and, if advantageous, can be immortalized for continuous growth in culture. Immortalization of B-lymphocytes isolated from chickens and their use in antibody production is described in U.S. Pat. No. 5,049,502 issued Sep. 17, 1991 to Humphries; U.S. Pat. No. 5,258,299 issued Nov. 2, 1993 to Humphries, and U.S. Pat. No. 6,143,559 issued Nov. 7, 2000 to Michael et al.

Another means by which homozygous deletions can be created in avian cells without the use of a second targeting step involves homogenization of the gene targeting event, as described in PCT application, PCT/US93/00926, herein incorporated in its entirety by reference. In this method, the targeting construct is introduced into a cell in a first targeting step, to create the desired genomic deletion. The cells are then screened for gene-targeted recombinants, and the recombinants are exposed to elevated levels of the selection agent for the marker gene, in order to select for cells which have multiple copies of the selective agent by other than amplification. The cells are then analyzed for homozygosity at the target locus.

DNA vectors may be employed which provide for the desired introduction of the targeting construct into the cell. The constructs may be modified to include functional entities other than the deletion targeting construct which may find use in the preparation of the construct, amplification, transfection of the host cell, integration of the construct into the host cell, and integration of additional sequences into the construct sequences when integrated into the host genome.

The replacement targeting construct may include a deletion at one site and an insertion at another site which includes a gene for a selectable marker. Of particular interest is a gene which provides a marker, e.g., antibiotic resistance such as neomycin resistance. The presence of the selectable marker gene inserted into the target gene establishes the integration of the target vector into the host genome. However, DNA analysis will be required in order to establish whether homologous or non-homologous recombination occurred. This can be determined by employing probes for the insert and then sequencing the 5′ and 3′ regions flanking the insert for the presence of DNA extending beyond the flanking regions of the construct or identifying the presence of a deletion, when such deletion is introduced. The selectable marker may be flanked by recombinase target site sequences, such that it can be excised by supplying an appropriate recombinase after selection of the transgenic cells and conformation of the homologously inserted sequence. Methods for excision of introduced sequences in transgenic cells using the cre-lox recombinase system is described in U.S. Pat. No. 6,066,778 issued May 23, 2000 to Ginsburg et al.

Upstream and/or downstream from the target gene construct may be a gene which provides for identification of whether a double crossover has occurred. For this purpose, the Herpes simplex virus thymidine kinase gene may be employed, since cells expressing the thymidine kinase gene may be killed by the use of nucleoside analogs such as acyclovir or gancyclovir, by their cytotoxic effects on cells that contain a functional HSV-tk gene. The absence of sensitivity to these nucleoside analogs indicates the absence of the HSV-thymidine kinase gene and, therefore, where homologous recombination has occurred, that a double crossover has also occurred.

Where a selectable marker gene is involved, as an insert, and/or flanking gene, depending upon the nature of the gene, it may be from a host where the transcriptional initiation region (promoter) is not recognized by the transcriptional machinery of the avian host cell. In this case, a different transcriptional initiation region (promoter) will be required. This region may be constitutive or inducible. A wide variety of transcriptional initiation regions have been isolated and used with different genes. Of particular interest is the promoter region of rous sarcoma virus. In addition to the promoter, the wild type enhancer may be present or an enhancer from a different gene may be joined to the promoter region.

While the presence of the marker gene in the genome will indicate that integration has occurred, it will still be necessary to determine whether homologous integration has occurred. This can be achieved in a number of ways. For the most part, DNA analysis will be employed to establish the location of the integration. By employing probes for the insert and then sequencing the 5′ and 3′ regions flanking the insert for the presence of the target locus extending beyond the flanking region of the construct or identifying the presence of a deletion, when such deletion has been introduced, the desired integration may be established.

The polymerase chain reaction (PCR) may be used with advantage in detecting the presence of homologous recombination. Probes may be used which are complementary to a sequence within the construct and complementary to a sequence outside the construct and at the target locus. In this way, one can only obtain DNA chains having both the primers present in the complementary chains if homologous recombination has occurred. By demonstrating the presence of the PCR products for the expected size sequence, the occurrence of homologous recombination is supported.

In constructing the subject constructs for homologous recombination, a replication system for procaryotes, particularly E. coli, may be included, for preparing the construct, cloning after each manipulation, analysis, such as restriction mapping or sequencing, expansion and isolation of the desired sequence. Where the construct is large, generally exceeding about 50 kbp, a yeast artificial chromosome (YAC) may be used for cloning of the construct. When necessary, a different selectable marker may be employed for detecting bacterial or yeast transformations.

Once a construct has been prepared and optionally, any undesirable sequences removed, e.g., procaryotic sequences, the construct may now be introduced into the target cell. Any convenient technique for introducing the DNA into the target cells may be employed. Techniques which may be used to introduce the replacement targeting construct into the avian cells include calcium phosphate/DNA coprecipitates, microinjection of DNA into the nucleus, electroporation, bacterial protoplast fusion with intact cells, transfection, particle gun bombardment, lipofection or the like. Where avian embryonic stem cells are used as the recipient cells, the DNA can be targeted to the cells using liposomes (Pain et al. Cells Tissues Organs 165: 212-219 (1999)). Where avian zygotes are used, the construct can be microinjected into the cytoplasm of the germinal disc (Love et al. Bio/Technology 12: 60-63 (1994). The DNA may be single or double stranded, linear or circular, relaxed or supercoiled DNA. After transformation or transfection of the target cells, target cells may be selected by means of positive and/or negative markers, as previously indicated, neomycin resistance and acyclovir or gancyclovir resistance. Those cells which show the desired phenotype may then be further analyzed by restriction analysis, electrophoresis, Southern analysis, PCR, or the like. By identifying fragments which show the presence of the lesion(s) at the target locus, one can identify cells in which homologous recombination has occurred to inactivate a copy of the target locus.

The above described process may be performed first with a heavy chain locus in an embryonic stem cell and then maturation of the cells to provide a mature fertile host. Then by breeding of the heterozygous hosts, a homozygous host may be obtained or embryonic stem cells may be isolated and transformed to inactivate the second Ig_{H} locus, and the process repeated until all the desired loci have been inactivated. Alternatively, the light chain locus may be the first. At any stage, the human loci may be introduced.

In one strategy, as individual steps, the constant regions of the avian heavy and light chain immunoglobulin gene complexes are rendered non-functional and in one or more separate steps one or more human constant region immunoglobulin genes are introduced into avian cells. Inactivation of the endogenous avian immunoglobulin loci is achieved by targeted disruption of the appropriate loci by homologous recombination inavian cells. Human heavy chain constant region and light chain constant region genes are reconstructed in an appropriate eukaryotic or prokaryotic microorganism and the resulting DNA fragments can be introduced into the avian cells. One or more or the eight human heavy chain immunoglobulin constant genes may be introduced into the avian cells. One human kappa light chain constant gene or one or more lambda light chain constant genes can be introduced into the avian cells. Where several genes are introduced together, the regions can be provided in one or more yeast artificial chromosomes (YACs). In another strategy, the human light and heavy chain loci are provided in targeting vectors that integrate into the avian light and heavy chain loci and thereby inactivate the endogenous loci.

These strategies are based on the known organization of the immunoglobulin chain loci in a number of animals, since the organization, relative location of exons encoding individual domains, and location of splice sites and transcriptional elements, is understood to varying degrees. In the human, the immunoglobulin heavy chain locus is located on chromosome 14. In the 5′-3′ direction of transcription, the locus comprises a large cluster of variable region genes (V_{H}), the diversity (D) region genes, followed by the joining (J_{H}) region genes and the constant (C_{H}) gene cluster. The size of the locus is estimated to be about 2,500 kilobases (kb). During B-cell development, discontinuous gene segments from the germ line Ig_{H} locus are juxtaposed by means of a physical rearrangement of the DNA. In order for a functional heavy chain Ig polypeptide to be produced, three discontinuous DNA segments, from the V_{H}, D, and J_{H} regions must be joined in a specific sequential fashion; V_{H} to DJ_{H}, generating the functional unit V_{H} DJ_{H}. Once a V_{H} DJ_{H} has been formed, specific heavy chains are produced following transcription of the Ig locus, utilizing as a template the specific V_{H} DJ_{H} C_{H} unit comprising exons and introns. There are two loci for Ig light chains, the kappa locus on human chromosome 2 and the .lambda. locus on human chromosome 22. The structure of the Ig_{L} loci is similar to that of the Ig_{H} locus, except that the D region is not present. Following Ig_{H} rearrangement, rearrangement of a light chain locus is similarly accomplished by V_{L} and J_{L} joining of the kappa or lambda chain. The sizes of the lambda and kappa loci are each approximately 1000 kb. Expression of rearranged Ig heavy chain and an Ig kappa or Ig lambda light chain in a particular B-cell allows for the generation of antibody molecules.

A preferred targeting construct for targeted disruption of the heavy chain constant region in avian is a vector containing a human heavy chain constant region gene, for example, a C gamma gene, and a selection marker such as the G418 or neomycin resistance gene. Similarly, for targeted disruption of the light chain constant region in avian, the target construct can contain a human light chain constant region gene, for example, the C kappa gene, and a second selection marker, which may be thymidine kinase (tk) or DHFR. Both of these vectors will contain 5′ and 3′ flanking sequence of the avian heavy and light chain constant genes flanking the human heavy and light chain constant genes, respectively. This would allow replacement of the human genes at the analogous position in the avian species.

It is important to target the human heavy and light chain constant genes to the chromosomal loci of the disrupted avian heavy and light chain constant genes. In this way the human constant regions will be placed in the same region as the analogous host constant region, so that recombination events (including gene rearrangements and gene conversion) associated with the generation of antibody diversity and the expression of functional chimeric antibodies can occur.

The construct carrying the exogenous immunoglobulin locus can therefore contain sequences of the endogenous avian immunoglobulin locus in order to promote homologous recombination of the exogenous immunoglobulin locus into the endogenous immunoglobulin locus. In another strategy, the gene disruption construct employed in inactivating the endogenous immunoglobulin loci can also transfer specific integration sequences to the disrupted locus. Sequences such as “lox”, “att”, and “frt” that allow highly efficient targeted integration of gene sequences can be introduced to these loci, and transient expression of the corresponding cre, int, or FLP recombinase can provide for efficient recombination of the introduced human sequences into the endogenous disrupted loci. Integration of the human sequences can occur simultaneously with excision of selectable marker sequences introduced into the locus by the gene disruption construct. Methods of using sequence-specific recombinase target sites and corresponding recombinases to fuse sequences or insert sequences is described in U.S. Pat. No. 4,959,317 issued Sep. 25, 1998 to Sauer, et al., U.S. Pat. No. 5,851,808 issued Dec. 22, 1998 to Elledge et al., U.S. Pat. No. 5,998,144 issued Dec. 7, 1999 to Reff et al., and U.S. Pat. No. 6,066,778 issued May 23, 2000 to Ginsburg et al., all herein incorporated by reference.

For the generation of transgenic avian cells the human DNA may be introduced into avian cells by any adequate method, including, for example, spheroplast fusion, lipofection, electroporation, calcium phosphate-mediated DNA transfer, particle gun bombardment, retroviral infection, or direct microinjection. The integration may be homologous or recombinase-mediated, depending on the particular strategy to be employed. For the generation of transgenic birds, replication-defective retroviruses can also be injected into the developing embryo (see, for example, U.S. Pat. No. 5,162,215 issued Nov. 10, 1992 to Bosselman et al.).

Transgenic birds carrying both targeted heavy chain constant regions and targeted light chain constant regions can be generated by selective breeding. For example, the modified avian cells with a human substituted heavy chain constant region a disrupted heavy chain locus can be used to generate transgenic avians that transmit the human heavy chain constant region through the germ line and modified avian cells with a human substituted light chain constant region can be used to generate transgenic avians that transmit the human light chain constant region through the germ line. Mating of avians that have human heavy chain constant regions to avians that have human light chain constant regions will produce progeny that have immunoglobulins with human heavy and light chain constant regions.

Once the human immunogloulin loci regions have been introduced into the avian host genome, either by homologous recombination, or the use of lox, att, or frt sequences, and host animals have been produced with the endogenous immunoglobulin loci inactivated by appropriate breeding of the various transgenic or mutated avians, one can produce an avian host which lacks the native capability to produce fully endogenous immunoglobulin subunits, but does hvae the capacity to produce human-avian chimeric immunoglobulins. Such a host strain, by immunization with specific antigens, would respond by the production of avian B-cells producing specific human-avian chimeric antibodies, which B-cells could be immortalized in any manner for the continuous stable production of human-avian chimeric monoclonal antibodies.

The subject methodology and strategies need not be limited to producing complete immunoglobulins, but provides the opportunity to provide for regions joined to a portion of the constant region, e.g., CH_{H1}, CH_{H2}, CH_{H3}, or CH_{H4}, or combination thereof. Alternatively, one or more of the exons of the C_{H} and C kappa or C lambda regions may be replaced or joined to a sequence encoding a different protein, such as an enzyme, e.g., plasminogen activator, superoxide dismutase, etc.; toxin A chain, e.g., ricin, abrin, diphtheria toxin, etc.; growth factors; cytotoxic agent, e.g., TNF, or a reporter protein, such as green fluorescent protein, beta galactosidase, beta-lactamase, alkaline phosphatase, or a specific binding protein or peptide such as glutathione-S-transferase, streptavidin, a series of histidine residues, or the like. See, for example, WO 89/07142; WO 89/09344; and WO 88/03559. By inserting the protein of interest into a constant region exon and providing for splicing of the variable region to the modified constant region exon, the resulting binding protein may have a different C-terminal region from the immunoglobulin. By providing for a stop sequence with the inserted gene, the protein product will have the inserted protein as the C-terminal region. If desired, the constant region may be entirely substituted by the other protein, by providing for a construct with the appropriate splice sites for joining the variable region to the other protein. Proteins useful in this regard include those listed above.

The antibodies or antibody analog producing B-cells from the transgenic host may be immortalized e.g., by transfection with oncogenes (see, for example, U.S. Pat. No. 6,143,559, issued Nov. 7, 2000 to Michael et al.). These immortalized cells may then be grown in continuous culture or introduced into the peritoneum of a compatible host for production of ascites. Immortalization of B-lymphocytes isolated from chickens described in U.S. Pat. No. 5,049,502 issued Sep. 17, 1991 to Humphries; U.S. Pat. No. 5,258,299 issued Nov. 2, 1993 to Humphries, and U.S. Pat. No. 6,143,559 issued Nov. 7, 2000 to Michael et al.

The subject invention provides for the production of polyclonal human-avian chimeric anti-serum or human-avian monoclonal antibodies or antibody analogs. Where the avian host has been immunized with an immunogen, the resulting chimeric antibodies can be isolated from other proteins by using an affinity column, having an Fc binding moiety, such as protein A, or the like.

Methods for Generating Transgenic Avians

When genetic loci of zygote cells from an avian host have been targeted, it may be desirable to use such cells to generate transgenic animals. For such a procedure, following the introduction of the targeting construct into the embryonic stem cells, the cells may be plated onto a feeder layer in an appropriate medium, for example, DMEM supplemented with growth factors and cytokines, fetal bovine serum and antibiotics (Pain et al. 1996 ). The embryonic stem cells may have a single targeted locus (heterozygotic) or both loci targeted (homozygotic). Cells containing the construct may be detected by employing a selective medium and after sufficient time for colonies to grow, colonies may be picked and analyzed for the occurrence of gene targeting. As described previously, PCR may be used, with primers within and outside the construct sequence, or Southern blot analysis or PFGE, but at the target locus. Those colonies which show gene targeting may then be used for injection into avian embryos. The ES cells can then be trypsinized and the modified cells can be injected through a an opening made in the side of the egg as described in U.S. Pat. No. 5,162,215. After sealing the eggs, the eggs can be incubated at 37 degrees C. until hatching. Newly hatched avians can be tested for the presence of the target construct sequences, for example by removing a blood sample. After the avians have reached maturity, they are bred and their progeny are examined to determine whether the gene targeting sequences are transmitted through the germ line.

Chimeric avians are generated which are derived in part from the modified embryonic stem cells or zygote cells, and are capable of transmitting the genetic modifications through the germ line. Mating avian strains containing human immunoglobulin loci, or portions thereof, to strains with strains in which the avian immunoglobulin loci, or portions thereof, have been deleted generates avians which produce chimeric or purely human antibodies.

Transgenic avians can also be other methods, some of which are discussed below. Among the avian cells suitable for transformation for generating transgenic animals are sperm cells, primordial germ cells, and zygote cells (including embryonic stem cells). Sperm cells can be transformed with DNA constructs by any suitable method, including electroporation, microparticle bombardment and lipofection (Gruenbaum et al. J. Cell. Biochem.15E, 194(1991); Rottman et al., J. Anim. Breed. Genet. 109: 64-70 (1992); Squires and Drake, Anim. Biotechnol. 4: 71-88 (1993). The sperm can be used for artificial insemination of avians. Progeny of the inseminated avian can be examined for the targeting sequence as described above.

Alternatively, genetically modified primordial germ cells (Petitte et al. Poult. Sci. 76: 1084-92 (1997) can be isolated from avian eggs (Vick et al., Proc. R. Soc. London Ser. B 251: 179-182 (1993); Tajima et al., Theriogenology 40: 509-519 (1993)), transfected with targeting constructs by any appropriate method, and transferred into new embryos, where they can become incorporated into the developing gonads. Hatched chicks and their progeny can be examined for the targeting sequence as described above.

In yet another approach, dispersed blastodermal cells isolated from eggs can be transfected by any appropriate means with a targeting construct or constructs containing exogenous immunoglobulin loci, or portions thereof, and injected into the subgerminal cavity of intact eggs (Carscience et al. Development 117: 669-75 (1993). Hatched chicks and their progeny can be examined for the targeting sequence as described above.

One of the advantages of the avian system is that the zygote is highly accessible to the researcher as it develops external to the female organism. For example, eggs containing developing zygotes can be injected with DNA constructs (Bosselman, R. A. et al., Science 243:533-535 (1989), and described in U.S. Pat. No. 5,162,215 ), or DNA can be introduced into cells of developing zygotes that are cultured outside the egg ( Perry, Nature 331: 70-72 (1988), Love et al. Bio/Technol. 12: 60-63 (1994), and Naito et al. Mol. Reprod. Dev. 37: 167-171 (1994) ). This is particularly useful where retroviral constructs are used, such as in the introduction of relatively small gene segments or recombination target sites.

In accordance with the above procedures, an avian host can be produced which can be immunized to produce avian-human chimeric antibodies or antibody analogs specific for an immunogen. In this manner, the problems associated with obtaining human monoclonal antibodies are avoided, since avians can be immunized with immunogens which could not be used with a human host. Furthermore, one can provide for booster injections and adjuvants, which would not be permitted with a human host. The resulting B-cells may then be used for immortalization for the continuous production of the desired antibody. The immortalized cells can optionally be used for isolation of the genes encoding the immunoglobulin or analog and can be reintroduced to other cell lines, including mammalian cell lines, for the production of antibody. Optionally, the genes can be subjected to mutation by in vitro mutagenesis or any other mutagenizing technique prior to reintroducing them to a cell line. Phage display methodologies can be used to select for nucleic acid sequences encoding immunoglobulins, or portions thereof, with modified properties (Davies, et al., J. Immunol. Methods 186: 125-135 (1995); and see also U.S. Pat. Nos. 5,223,409, 5,846,533, and 5,824,520, all herein incorportated by reference). These mutagenized nucleic acid sequences may then be returned to an immortalized cells to provide for a continuous avian cellular source of the desired antibodies or antibody analogs. The subject invention provides for a convenient source of avian-human chimeric antibodies, where the avian-human chimeric antibodies are produced in analogous manner to the production of antibodies in a human host.

Avian Cells for Producing Chimeric Antibodies

In another embodiment of the present invention, avians are challenged with an antigen of interest and tested for the production of antibodies reactive against the antigen of interest. The avians of the present invention can be of any avian species, such as but not limited to, ducks, geese, turkeys, and quails, but are preferably chickens. Avians producing the antigen of interest are used for the isolation of B-lymphocytes which are immortalized by any appropriate method, for example, the introduction of an oncogene. Immunization of avians, isolation of B-lymphocytes from avians, and immortalization of B-lymphocytes isolated from avians are described in Michael et al. Proc. Natl. Acad. Sci. USA 95: 1166-1171 (1995), U.S. Pat. No. 5,049,502, U.S. Pat. No. 5,258,299, and U.S. Pat. No. 6,143,559, all herein incorporated by reference.

The cells are tested again for the production of antibody reactive against the antigen of interest. Positively screening clones are selected for gene targeting, such that the endogenous constant heavy chain and light chain immunoglobulin regions are replaced with exogenous constant heavy chain and light chain immunoglobulin regions.

The present invention includes the generation of genomic DNA deletions or gene disruptions in avian cells. The method of the invention provides the use of a replacement-type targeting construct to delete fragments of genomic DNA by gene targeting. Methods of creating non-human transgenic mammals using gene targeting are described in U.S. Pat. No. 5,998,209 issued Dec. 7, 1999 to Jakobovits, et al., U.S. Pat. No. 6,066,778 issued May 23, 2000 to Ginsburg et al., all herein incorportated by reference. Methods for generating non-human transgenic mammals lacking a functional endogenous immunoglobulin locus and carrying a functional exogenous, preferably human, immunoglobulin locus are described in U.S. Pat. No. 5,939,598 issued Aug. 17, 1999 to Kucherlapati et al., and PCT WO 94/02602, both herein incorporated by reference. The replacement targeting construct, which may contain a selectable marker, is constructed to contain two regions of sequences which are homologous to the 5′ and 3′ flanking sequences of the targeted locus. After transfection of the targeting construct into the desired cell line, gene targeted-mediated deletions may be identified by selection and further characterized by PCR, Southern blot analysis, and/or pulsed field gel electrophoresis (PFGE).

The transgenic avian cells can be used to produce chimeric, preferably human-avian antibodies, or modified antibodies. Genomic deletions or gene disruptions are created in the constant regions of endogenous immunoglobulin loci in avian cells, and concurrently or in separate steps, the human heavy and light chain immunoglobulin gene constant regions are introduced into the avian genome. This is accomplished by reconstructing the human heavy and light chain immunoglobulin gene constant regions, or portions thereof, in an appropriate eukaryotic or prokaryotic microorganism and introducing the resulting DNA fragments into avian cells. The chimeric antibody or modified antibody producing immortalized B-cells from the transgenic host can then be grown in continuous culture or introduced into the peritoneum of a compatible host for production of ascites.

The subject invention provides for the production of human-avian chimeric monoclonal antibodies or antibody analogs. The resulting chimeric antibodies may be isolated from other proteins by using an affinity column, having an Fc binding moiety, such as protein A, or the like.

### EXAMPLES

### Example I: Inactivation of the Chicken Heavy Chain J Genes

Construction of the Inactivation Vector

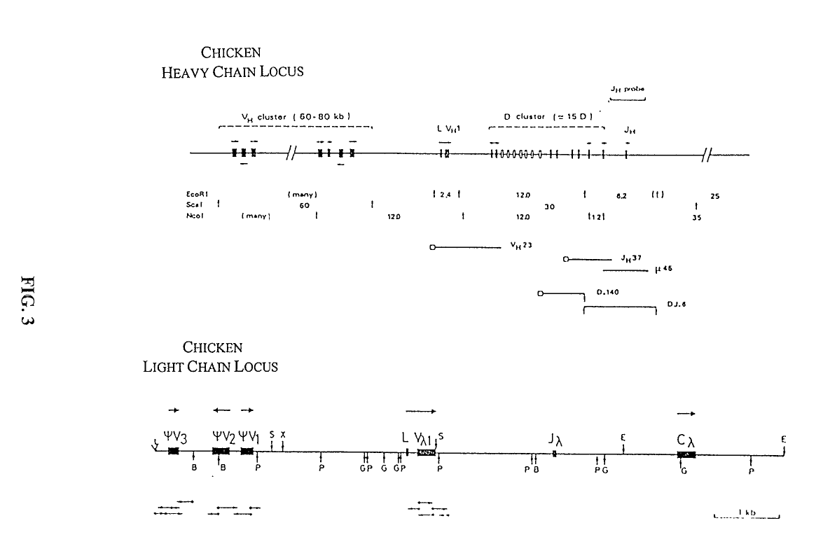

A 4.5 Kb fragment, containing the chicken heavy chain J genes and flanking sequences, is PCR amplified from a White Leghorn chicken strain genomic library (Reynaud et al., 1989) containing Eco RI cloning sites in the PCR primers and inserted into EcoRI-digested pUC19 plasmid (pchkJ_{H}) (see FIG. 3 for chicken heavy gene complex). An 1150 bp Xho I-Bam HI fragment, containing a neomycin-resistance gene driven by the Herpes simplex virus thymidine kinase gene (HSV-tk) promoter and a polyoma enhancer is isolated from pMClNeo (Thomas and Capecchi, Cell, 51, 503-512, 1987). A synthetic adaptor is added onto this fragment to convert the Barn HI end into a Sca I end and the resulting fragment is joined to the Xho I-Sca I digested PchkJ_{H} to form the inactivation vector (pchkJ.Neo) in which the heavy chain J genes are excised, and the 5′ to 3′ orientation of the neomycin and the heavy chain promoters is identical. This plasmid is linearized by Nde I digestion before transfection into ES cells. The sequences driving the homologous recombination event are 3 kb and 0.5 kb fragments from the D cluster region of the heavy chain gene upstream of the heavy chain J gene and from sequences downstream of the heavy chain J gene, and located 5′ and 3′ to the neomycin gene, respectively.

Isolation and Culture of Chicken ES Cells

The ES cells are isolated from blastodermal cells, maintained and amplified in vitro (Pain et al., 1996). The entire blastoderm from embryos of White Leghorn chickens at stages IX-XI is removed by gentle aspiration with a Pasteur pipette in PBS containing 5.6 mM D-glucose (PBS-G) at room temperature. Embryos are pooled at 1 embryo per ml and centrifuged at 400 g twice. The cell pellet is then slowly mechanically dissociated in ESA medium (Glasgow-MEM, containing 105 fetal bovine serum, 2% chicken serum, 1% bovine serum albumin, 20 ng/ml conalbumin, 1 mM sodium pyruvate, 1% non-essential amino acids, 1 mM of each of the nucleotides adenosine, guanosine, cytidine, uridine, thymidine, 10 mM Hepes, pH 7.6, 0.16 mM beta-mercaptoethanol, 100 U/ml penicillin, 100 mg/ml streptomycin, and 10 ng/ml gentamycin). Cells are seeded in ESA complete medium (ESA medium supplemented with 10 ng/ml bFGF, 20 ng/ml h-IGF-1, 1% vol/vol avian SCF and 1% vol/vol h-LIF, 1% v/v h-IL-11) on gelatin precoated dishes or inactivated STO feeder cells. The blastodermal cells are maintained at 37 degrees C. in 7.5% CO₂ and 90% humidity. Half of the medium is replaced after 24 hrs in culture. Fresh blastodermal cells are added in half of the original volume of ESA complete medium 48 hr later. The medium is changed partially (50%) on the third day and totally every day thereafter. The cells are recovered by washing the cells in PBS-G and incubating in a solution of pronase (0.025% w/v).

Transfection and Screening of Chicken ES Cells

The chicken ES cells (CES) derived as above are transfected with J_{H} inactivating vector, pchkJ vector using a transfection reagent. A lipid based agent, FuGENE6 (Roche Bioproducts) has been shown to be an optimal reagent for introducing exogenous DNA into CES cells. The transfected cells are seeded on the new feeder cells or on gelatin-coated dishes in complete ESA medium containing G418 for selection of stable transfectants.

ES colonies remaining 10-14 days after transfection are picked with drawn out capillary pipettes for analysis using PCR. Half of each picked colony is saved in 24-well plates already seeded with mitomycin-treated feeder cells. The other halves, combined in pools of 3-4, are transferred to Eppendorf tubes containing approximately 0.5 ml of PBS and analyzed for homologous recombination by PCR. Conditions for PCR reactions are essentially as described (Kim and Smithies, Nucleic Acids Res. 16:8887-8893, 1988). After pelleting, the CES cells are resuspended in 5 ml of PBS and are lysed by the addition of 55 ml of H₂O to each tube. DNAses are inactivated by heating each tube at 95° C. for 10 min. After treatment with proteinase K at 55° C. for 30 min, 30 microliters of each lysate is transferred to a tube containing 20 microliters of a reaction mixture including PCR buffer: 1.5 micrograms of each primer, 3U of Taq polymerase, 10% DMSO, and dNTPs, each at 0.2 mM. The PCR expansion employs 55 cycles using a thermocycler with 65 seconds melting at 92 degrees C. and a 10 min annealing and extension time at 65 degrees C. One priming oligonucleotide corresponds to a region 650 bases 3′ of the start codon of the neomycin resistance gene and the other priming oligonucleotide corresponds to sequences located in the human heavy chain gene that are outside the region of homology included in the targeting vector. Twenty microliters of each reaction mix is electrophoresed on agarose gels and transferred to nylon membranes (Zeta Bind). Filters are probed with a ³²P-labeled fragment of the J-C region. Because the PCR primers employed will only amplify a segment of DNA in which the DNA neomycin-resistance gene is physically linked to PCR products that hybridize to the probe, hybridizing PCR products of the expected size are derived from loci in which the neomycin gene has homologously recombined into the J region of the heavy chain locus, thereby inactivating the locus.

### Example II: Inactivation of the Chicken Ig Light Chain J Genes in ES Cells

Construction of the Inactivation Vector

A 4.5 Kb fragment, containing the chicken immunoglobulin light chain J region genes and flanking sequences is amplified by PCR from a chicken genomic library using PCR primers containing Eco RI cloning sites and inserted into pUC18 (pchkJ_{L}) (see FIG. 3 for chicken light gene complex). An about 1.1 kbp Xho I-Bam HI fragment, blunted at the Bam HI site, containing a neomycin resistance gene driven by the Herpes simplex virus thymidine kinase gene (HSV-tk) promoter and polyoma enhancer was isolated from pMClNeo (Thomas and Capecchi, Cell, 51, 503-512, 1987). This fragment was inserted into the XhoI-NaeI deleted PJ_{L} to form the inactivation vector (pchk J_{L}), in which the J genes are excised and the transcriptional orientation of the neomycin and the light chain genes is the same. This plasmid was linearized by Nde I digestion before transfection to ES cells. The sequences driving the homologous recombination event are about 2.8 kbp and about 1.1 kbp fragments, from the region of the lambda light chain gene upstream of the J region and downstream of the light chain J gene, and located 5′ and 3′ to the neomycin gene, respectively.

The ES cells are isolated from blastodermal cells, maintained and amplified in vitro (Pain et al., 1996). The entire blastoderm from embryos of White Leghorn chickens at stages IX-XI is removed by gentle aspiration with a Pasteur pipette in PBS containing 5.6 mM D-glucose (PBS-G) at room temperature. Embryos are pooled at 1 embryo per ml and centrifuged at 400 g twice. The cell pellet is then slowly mechanically dissociated in ESA medium (Glasgow-MEM, containing 105 fetal bovine serum, 2% chicken serum, 1% bovine serum albumin, 20 ng/ml conalbumin, 1 mM sodium pyruvate, 1% non-essential amino acids, 1 mM of each of the nucleotides adenosine, guanosine, cytidine, uridine, thymidine, 10 mM Hepes, pH 7.6, 0.16 mM beta-mercaptoethanol, 100 U/ml penicillin, 100 mg/ml streptomycin, and 10 ng/ml gentamycin). Cells are seeded in ESA complete medium (ESA medium supplemented with 10 ng/ml bFGF, 20 ng/ml h-IGF-1, 1% vol/vol avian SCF and 1% vol/vol H-LIF, 1% v/v h-IL-11) on gelatin precoated dishes or inactivated STO feeder cells. The blastodermal cells are maintained at 37 degrees C. in 7.5% CO₂ and 90% humidity. Half of the medium is replaced after 24 hrs in culture. Fresh blastodermal cells are added in half of the original volume of ESA complete medium 48 hr later. The medium is changed partially (50%) on the third day and totally every day thereafter. The cells are recovered by washing the cells in PBS-G and incubating in a solution of pronase (0.025% w/v).

Transfection and Screening of Chicken ES Cells

The chicken ES cells (CES) derived as above are transfected with J_{L} inactivating vector, pchkJ_{L}, vector using a transfection reagent. A lipid based agent, FuGENE6 (Roche Bioproducts) has been shown to be an optimal reagent for introducing exogenous DNA into CES cells. The transfected cells are seeded on the new feeder cells or on gelatine-coated dishes in complete ESA medium containing G418 for selection of stable transfectants.

ES colonies remaining 10-14 days after transfection are picked with drawn out capillary pipettes for analysis using PCR. Half of each picked colony is saved in 24-well plates already seeded with mitomycin-treated feeder cells. The other halves, combined in pools of 3-4, are transferred to Eppendorf tubes containing approximately 0.5 ml of PBS and analyzed for homologous recombination by PCR. Conditions for PCR reactions are essentially as described (Kim and Smithies, Nucleic Acids Res. 16:8887-8893, 1988). After pelleting, the CES cells are resuspended in 5 ml of PBS and are lysed by the addition of 55 ml of H₂O to each tube. DNAses are inactivated by heating each tube at 95° C. for 10 min. After treatment with proteinase K at 55° C. for 30 min. 30 microliters of each lysate is transferred to a tube containing 20 microliters of a reaction mixture including PCR buffer: 1.5 micrograms of each primer, 3U of Taq polymerase, 10% DMSO, and dNTPs, each at 0.2 mM. The PCR expansion employs 55 cycles using a thermocycler with 65 seconds melting at 92 degrees C. and a 10 min annealing and extension time at 65 degrees C. One priming oligonucleotide corresponds to a region 650 bases 3′ of the start codon of the neomycin resistance gene and the other priming oligonucleotide corresponds to sequences located in the human heavy chain gene that are outside the region of homology included in the targeting vector. Twenty microliters of each reaction mix is electrophoresed on agarose gels and transferred to nylon membranes (Zeta Bind). Filters are probed with a ³²P-labeled fragment of the J-C region. Because the PCR primers employed will only amplify a segment of DNA in which the DNA neomycin-resistance gene is physically linked to PCR products that hybridize to the probe, hybridizing PCR products of the expected size are derived from loci in which the neomycin gene has homologously recombined into the J region of the light chain locus, thereby inactivating the locus.

### Example III: Production of Human Heavy Chain Immunoglobulin in Transgenic Chicken

Cloning of the Human Heavy Chain Immunoglobulin in a YAC Vector

An Spe I fragment, spanning the human heavy chain VH6-D-J-Cm Cd region (Berman et al., EMBO J. (1988) 7: 727-738; see FIG. 3A) is isolated from a human library cloned into a yeast artificial chromosome (YAC) vector (Burke, et al., Science, 236: 806-812) using DNA probes described by Berman et al. (EMBO J. (1988) 7:727-738). One clone is obtained which is estimated to be about 100 Kb. The isolated YAC clone is characterized by pulsed-field gel electrophoresis (Burke et al., supra; Brownstein et al., Science, 244: 1348-13451), using radiolabelled probes for the human heavy chain (Berman et al., supra).

Introduction of YAC Clones into Embryos

High molecular weight DNA is prepared in agarose plugs from yeast cells containing the YAC of interest (i.e., a YAC containing the aforementioned Spe I fragment from the Ig_{H} locus). The DNA is size-fractionated on a CHEF gel apparatus and the YAC band is cut out of the low melting point agarose gel. The gel fragment is equilibrated with polyamines and then melted and treated with agarase to digest the agarose. The polyamine-coated DNA is then injected into the blastoderm of fertilized chicken egg. The transgenic nature of the hatchlings is analyzed by a slot-blot of DNA isolated from blood cells and the production of human heavy chain is analyzed by obtaining a small amount of serum and testing it for the presence of Ig chains with rabbit anti-human antibodies.

As an alternative to microinjection, YAC DNA is transferred into CES cells by ES cell: yeast protoplast fusion (Traver et al., 1989 Proc. Natl. Acad. Sci., USA, 86:5898-5902; Pachnis et al., 1990, ibid 87: 5109-5113). First, the neomycin-resistance gene from pMClNeo and a yeast selectable marker are inserted into nonessential YAC vector sequences in a plasmid. This construct is used to transform a yeast strain containing the IgH YAC, and pMClNeo is integrated into vector sequences of the IgH YAC by homologous recombination. The modified YAC is then transferred into an ES cell by protoplast fusion (Traver et al., 1989; Pachnis et al., 1990), and resulting G418-resistant ES cells which contain the intact human IgH sequences are used to generate chimeric chicken.

### Example IV: Production of Human Ig By Chimeric Chicken

Construction of Human Light Chain Replacement Vector

As an alternative to separately disrupting the chicken immunoglobulin locus and introducing human immunoglobulin genes into the chicken, this vector will allow complete replacement of chicken heavy chain complex including yV_{H} cluster, V_{H1}, D cluster, J_{H}, and Cm genes with human V genes, D, J_{H}, Cm, and Cd genes. The replacing human sequences include the Spe I 100 kbp fragment of genomic DNA which encompasses the human VH6-D-J-CmCd heavy chain region isolated from a human YAC library as described before. The flanking chicken heavy chain sequences, which drive the homologous recombination replacement event, contain a fragment of the chicken Cm chain sequences and a fragment comprising a fragment of the chicken V_{H}, at the 3′ and 5′ ends of the human sequences, respectively (FIG. 3B). These chicken sequences are isolated from a chicken genomic library using the probes described in (Reynaud et al., 1989). The 1150 bp Xho I to Bam HI fragment, containing a neomycin-resistance gene driven by the Herpes simplex virus thymidine kinase gene (HSV-tk) promoter and a polyoma enhancer is isolated from pMClNeo (Koller and Smithies, 1989, supra). A synthetic adaptor is added onto this fragment to convert the Xho I end into a Bam HI end and the resulting fragment is joined to the Bam HI site in the chicken Cm region sequences in a plasmid.

From the YAC clone containing the human heavy chain locus, DNA sequences from each end of the insert are recovered either by inverse PCR (Silverman et al., PNAS, 86:7485-7489, 1989), or by plasmid rescue in E. coli, (Burke et al., 1987; Garza et al. Science, 246:641-646, 1989; Traver et al., 1989). The isolated human sequence from the 5′V6 end of the YAC is ligated to chicken V_{H} sequence in a plasmid and likewise, the human sequence derived from the 3Cd end of the YAC is ligated to the Neo gene in the plasmid containing Neo and chicken Cm described above. The human V6- chicken V_{H} segment is now subcloned into a half-YAC cloning vector that includes a yeast selectable marker (HIS3) not present in the original IgH YAC, a centromere (CEN) and a single telomere (TEL). The human Cd Neo- chicken Cm is likewise subcloned into a separate half-YAC vector with a different yeast selectable marker (LEU2) and a single TEL. The half-YAC vector containing the human V6 DNA is linearized and used to transform a yeast strain that is deleted for the chromosomal HIS3 and LEU2 loci and which carries the IgH YAC. Selection for histidine-prototrophy gives rise to yeast colonies that have undergone homologous recombination between the human V6 DNA sequences and contain a recombinant YAC. The half-YAC vector containing the human Cd DNA is then linearized and used to transform the yeast strain generated in the previous step. Selection for leucine-prototrophy results in a yeast strain containing the complete Ig_{H} replacement YAC. This YAC is isolated and introduced into embryos by microinjection as described previously for eggs or by protoplast fusion with chicken ES cells.

Construction of Human Light Chain Replacement Vector

This vector will allow complete replacement of chicken light chain complex including yV₁ cluster, V₁₁, J, and C1 genes with human V genes, J, C₁, or Cₖ genes. The constructs would be made as described above. However, the human heavy chain gene components will be replaced by human light chain components. The IgL YAC is isolated and introduced into embryos by microinjection as described previously for eggs or by protoplast fusion with chicken ES cells.

All publications, including patent documents and scientific articles, referred to in this application, including any bibliography, are incorporated by reference in their entirety for all purposes to the same extent as if each individual publication were individually incorporated by reference. All headings are for the convenience of the reader and should not be used to limit the meaning of the text that follows the heading, unless so specified.

### BIBLIOGRAPHY

4816397 March, 1989 Boss et al.

4816567 March, 1989 Cabilly et al.

4950599 August, 1990 Bertling

4959313 September, 1990 Taketo

4975369 December, 1990 Beavers et al.

5,028,540 July, 1991 Humphries et al.

5,049,502 September, 1991 Humphries et al.

5202238 April, 1993 Fell et al.

5,258,299 November, 1993 Humphries et al.

5204244 April, 1993 Fell

5340740 August, 1994 Petitte et al.

5545806 August, 1996 Lariberg

5591669 January, 1997 Krimpenfnt

5643745 July, 1997 Stuart.

5656479 August, 1997 Petitte et al.

5683899 November, 1997 Stuart.

5695965 December, 1997 Stuart et al.

5,916,771 Jun. 29, 1999 Hori et al.

5,939,598 Aug. 17, 1999 Kucherlapati et al.

5,998,209 Dec. 7, 1999 Jakobovitis et al.

6,114,598 Sep. 5, 2000 Kucherlapati et al.

6,162,963 Dec.19, 2000 Kucherlapati et al.

9110741 July, 1991 WO.

0 273 889 July, 1988 EP.

0 088 994 B1 June, 1991 EP.

WO 86/01533 March, 1986 WO.

WO 92/03918 March, 1992 WO.

WO 92/15322 September, 1992 WO.

WO 93/19172 September, 1993 WO.

WO 93/25663 December, 1993 WO.

WO 94/02602 February, 1994 WO.

WO 95/02686 January, 1995 WO.

WO 95/30739 November, 1995 WO.

Bebbington, C. R., (1991) “Expression of Antibody Genes in Nonlymphoid Mammalian Cells,” Methods: A Companion to Methods Enzymol. 2:136-145.

Bebbington, C. R., et al., (1992) “High-level Expression of a Recombinant Antibody from Myeloma Cells Using a Glutamine Synthetase Gene As An Amplifiable Selectable Marker,” Bio/Technology 10:169-175.

Berman et al.; EMBO Journal, 7(3):727-738 (1988); Content and Organization of the human Ig V.sub.H locus: definition of three new V.sub.H families and linkage to the Ig C.sub.H locus.

Blankenstein and Kruwinkel; Eur. J. Immunol. 17:1351-1357 (1987). Joyner et al.; Nature 338:153-155 (1989).

Bos, et al., “Enhanced Transfusion of a Bacterial Plasmid of Hybrid Hybridoma (Quadroma Cell) Lines,” Hybridoma (February 1992), vol. 11, No. 1, pp. 41-51.

Brownstein et al.; Science 244:1348-1351 (1989); Isolation of Single-Copy Human Genes from a Library of Yeast Artificial Chromosome Clones.

Bruggemann et al. PNAS 86: 6709, 1989.

Bruggemann et al. Proc. Natl. Acad. Sci. USA 86:6709-6713 (1989); A Repertoire of Monoclonal antibodies with Human Heavy Chains from Transgenic Mice.

Burke et al. Science, 236:806-812 (1987); Cloning of Large Segments of Exogenous DNA into Yeast by Means of Artifical Chromosome Vectors.

Buttin G.; Trends in Genetics 3(8):205-206 (1987); Exogenous Ig Gene Rearrangement in Transgenic Mice: A New Strategy for Human Monocloanl Antibody Production.

Cao, et al., “A Rapid Non-Selective Method to Generate Quadromas by Microelectrofusion,” J. Immunol. Methods (Nov. 16, 1995), vol. 187, No. 1, pp. 1-7.

Capecchi et al., “Altering The Genome By Homologous Recombination,” Science, 244, pp. 1288-1292 (1989).

Caron, P. C., et al., (1992) “Engineered Humanized Dimeric Forms of IgG Are More Effective Antibodies,” J. Exp. Med. 176:1191-1195.

Cattaneo, A. and Neuberger, M. S., (1987) “Polymeric Immunoglobulin M Is Secreted by Transfectants of Non-lymphoid Cells in the Absence of Immunoglobulin J Chain,” EMBO J. 6:2753-2758.

Choi et al., “RNA Splicing Generates A Variant Light Chain From An Aberrantly Arranged kappa. Gene,” Nature, 286, pp. 776-779 (1980).

Cockett, M. I., et al., (1990) “High-level Expression of Tissue Inhibitor of Metalloproteinases in Chinese Hamster Ovary Cells Using Glutamine Synthetase Game Amplification,” Bio/Technology 8:662-667.

Cook et al. Nat Genet 7: 162-8 (1994).

Davies et al., J. Immunol. Methods 186: 125-135 (1995).

Doetschman et al., “Targeted Mutation Of The Hprt Gene in Mouse Embryonic Stem Cells,” Proc. Natl. Acad. Sci., 85, pp. 8583-8587 (1988).

Dorfman, Nickolas A., 1985, “The Optimal Technological Approach to the Development of Human Hybridomas,” Journal of Biological Response Modifiers, 4:213-239.

Garza et al.; Science 246:641-646 (1989); Mapping the Drosophila Genome with Yeast Artificial Chromosomes.

Graham, F. L., and Vander Eb, A. J., (1973) “A New Technique for the Assay of Infectivity of Human Adenovirus 5 DNA,” Virology 52:456-467.

Johnson et al., “Targeting Of Nonexpressed Genes in Embryonic Stem Cells Via Homologous Recombination,” Science, 245, pp. 1234-1236 (1989).

Koller et al.; Proc. Natl. Acad. Sci. USA, vol. 86:8932-8935 (1989); Inactivating the .beta.sub.2 -microglobulin locus in mouse embryonic stem cells by homologous recombination.

Kranenborg, et al., “Development and Characterization of Anti-Renal Cell Carcinoma X Antichelate Bispecific Monoclonal Antibodies for Two-Phase Targeting of Renal Cell Carcinoma,” Cancer Research (December 1995), vol. 55, No. 23, pp. 5864S-5867S.

Kucherlapati et al., “Homologous recombination in mammalian somatic cells,” Prog. Nucleic Acid Res. Mol. Biol., 36, pp. 301-310 (1989).

Mansour et al., “Disruption of the Proto-oncogene Int-2 In Mouse Embryo-derviced Stem Cells: A General Stratgegy For Targeting Mutations To Non-selectable Genes,” Nature, 336, pp. 348-352 (1988).

Max et al., “Sequences of Five Potential Recombination Sites Encoded Close To An Immunoglobulin .kappa. Constant Region Gene,” Proc. Natl. Acad. Sci., 76, pp. 3450-3454 (1979).

Mohammed et al. Immunotechnology 4: 115-25 (1998).

Orkin et al., “Mutation In An Intervening Sequence Splice Junction In Man,” Proc. Natl. Acad. Sci., 78, pp. 5041-5045 (1981).

Pain et al. Development 122: 2339-2348 (1996).

Pain et al. Cells Tissues Organs 165: 212-219 (1999).

Rajewski et al., “Evolutionary and Somatic Selection of the Antibody Repertoire in the Mouse,” Science, 238, pp. 1088-1094 (1987).

Ramirez-Solis et al., “Chromosome Engineering In Mice,” Nature, 378, pp. 720-724 (1995).

Reynaud et al. Cell 40: 283-291 (1985).

Riechmann, L. et al., (1988) “Reshaping Human Antibodies for Therapy,” Nature 332:323-327.

Sakano et al., “Sequences At The Somatic Recombination Sites Of Immunoglobulin Light-Chain Genes,” Nature, 280, pp. 288-294 (1979).

Sakano et al., “Two Types Of Somatic Recombination Are Necessary For The Generation Of Complete Immunoglobulin Heavy-Chain Genes,” Nature, 280, pp. 676-683 (1980).

Sakano et al.; Nature 290:562-565 (1981); Identification and Nucleotide Sequence of a Diversity DNA Segment (D) of Immunoglobulin Heavy-Chain Genes.

Salazar-Kish, et al., “Comparison of a Quadroma and its Parent Hybridomas in Fed Batch Culture,” J. Biotechnology (1993), vol. 30, No. 3, pp. 351-365.

Schwartzberg et al., “Germ-line Transmission Of A c-abl Mutation Produced By Targeted Gene Disruption In ES Cells,” Science, 246, pp. 799-803 (1989).

Seidman and Leder, “A Mutant Immunoglobulin Light Chain Is Formed By Aberrant DNA-and RNA-Splicing Events,” Nature, 286, pp. 779-783 (1980).

Shimizu, et al., PNAS USA (1989) 86, 8020-8023.

Taggart et al., 1983, “Stable Antibody-Producing Murine Hybridomas,” Science 219:1228-1230.

Thomas et al. “Site-Directed Mutagenesis by Gene Targeting in Mouse Embryo-Derived Stem Cells” Cell 51: 503-512 (1987).

Traver et al.; Proc. Nat. Acad. Sci. USA 86:5898-5902 (1989). Panchis et al. Proc. Nat. Acad. Sci. USA 87:5109-5113 (1990).

Treisman et al., “Specific Transcription and RNA Splicing Defects In Five Cloned .beta.-Thalassaemia Genes,” Nature, 302, pp. 591-596 (1983).

Tucker et al.; Proc. Natl. Acad. Sci. USA 78(12):7684-7688 (1981); Mouse IgA Heavy Chain Gene Sequence: Implications for Evolution of Immunoglobulin Hinge Exons.

Wood, et al., “High Level Synthesis of Immunoglobulins in Chinese Hamster Ovary Cells,” J. Immunol. 145(9):3011-3016 (Nov. 1, 1990).

Yamamura, et al., PNAS USA (1986) 83: 2152-2156.

Yancopoulos et al.; Science 241: 1581-1583 (1988); Reconstruction of an Immune System.

Zhang et al, Molecular and Cellular Biology vol. 14(4): pp. 2404-2410 (April 1994).

Zjilstra et al., “Germ-line Transmission Of A Disrupted .beta.2-Microglobulin Gene Produced By Homologuous Recombination In Embryonic Stem Cells,” Nature, 342, pp. 435-438 (1989).

We claim:

1. A method of making a transgenic avian lacking expression of endogenous immunoglobulin, comprising:
inactivating at least one endogenous heavy chain immunoglobulin locus in at least one avian cell;
generating at least one avian from said at least one avian cell; and
optionally breeding said at least one avian to obtain a transgenic avian lacking expression of endogenous immunoglobulins.

2. The method of claim 1, further comprising introducing at least a portion of at least one exogenous immunoglobulin locus into at least one avian cell.

3. The method of claim 2, wherein said at least a portion of said at least one exogenous immunoglobulin locus comprises at least a portion of at least one heavy chain constant region.

4. The method of claim 3, wherein said at least one heavy chain constant region is a human heavy chain constant region.

5. The method of claim 3, wherein said at least a portion of at least one exogenous immunoglobulin locus comprises at least a portion of the V_{H}, D_{H}, J_{H} and C_{H} regions.

6. The method of claim 1, further comprising inactivating at least one endogenous immunoglobulin light chain locus in at least one avian cell.

7. The method of claim 6, further comprising introducing at least a portion of at least one exogenous immunoglobulin light chain locus into at least one avian cell.

8. The method of claim 7, wherein said at least a portion of at least one exogenous immunoglobulin light chain locus is at least a portion of at least one human immunoglobulin light chain locus.

9. The method of claim 7, wherein said at least a portion of at least one exogenous immunoglobulin light chain locus comprises at least a portion of at least one light chain constant region.

10. The method of claim 7, wherein said at least a portion of at least one exogenous immunoglobulin light chain locus comprises at least a portion of the V_{L}, J_{L}, and C_{L} regions.

11. The method of claim 1, wherein said avian cell is a chicken cell, a turkey cell, a duck cell, a goose cell, or a quail cell.

12. A method of making a chimeric antibody, comprising:
immunizing the transgenic avian of claim 3 with an antigen;
harvesting serum or obtaining at least one egg from said transgenic avian; and
isolating at least one chimeric antibody or at least one exogenous antibody from said serum or said at least one egg.

13. An antibody made by the method of claim 12.

14. A method of making a chimeric monoclonal antibody, comprising:
immunizing the transgenic avian of claim 3 with an antigen;
harvesting B cells from said transgenic avian;
immortalizing said B cells; and
isolating at least one monoclonal antibody from the culture medium of said B cells.

15. An antibody made by the method of claim 14.

16. A method of making a xenogenic antibody, comprising:
immunizing the transgenic avian of claim 10 with an antigen;
harvesting serum or obtaining at least one egg from said transgenic avian; and
isolating at least one xenogenic antibody from said serum or said at least one egg.

17. An antibody made by the method of claim 16.

18. A method of making a xenogenic monoclonal antibody, comprising:
immunizing the transgenic avian of claim 10 with an antigen;
harvesting B cells from said transgenic avian;
immortalizing said B cells; and
isolating at least one monoclonal antibody from the culture medium of said B cells.

19. An antibody made by the method of claim 18.

20. The method of claim 18, further comprising:
isolating at least one nucleic acid molecule comprising cDNA encoding at least a portion of an immunoglobulin from said immortalized B cells;
introducing said at least one nucleic acid molecule comprising cDNA encoding at least a portion of an immunoglobulin into at least one other cell;
culturing said at least one other cell under conditions that promote protein synthesis; and
isolating at least one antibody from the culture medium of said at least one other cell.

21. The method of claim 20, wherein said at least one other cell is at least one prokaryotic, fungal, avian or mammalian cell.

22. An antibody made by the method of claim 20.
